(19) United States
(12) Patent Application Publication
(10) Pub. No.: US 2026/0272934 A1
(43) Pub. Date: Sep. 17, 2026

(54) 1-THIOXO-2,4-DIHYDROTHIENO[2,3-E] [1,2,4]TRIAZOLO[4,3-A]PYRIMIDIN-5(1H)-ONE DERIVATIVES AS ALLOSTERIC INHIBITORS OF THE POLO-LIKE KINASE 1 (PLK1) POLO BOX DOMAIN (PBD) FOR THE TREATMENT OF CANCER

(71) Applicant: The United States of America, as represented by the Secretary, Department of Health and Human Servic, Bethesda, MD (US)

(72) Inventors: Kyung S. Lee, Gaithersburg, MD (US); Kenneth A. Jacobson, Silver Spring, MD (US); Jung-Eun Park, North Bethesda, MD (US); Hobin Lee, Bethesda, MD (US); Klara Pongorne Kirsch, Bethesda, MD (US)

(73) Assignee: The United States of America, as represented by the Secretary, Department of Health and Human Servic, Bethesda, MD (US)

(21) Appl. No.: 19/168,983

(22) PCT Filed: Mar. 29, 2024
(86) PCT No.: PCT/US2024/022328

§ 371 Date: Sep. 25, 2025

Related U.S. Application Data
(60) Provisional application No. 63/455,608, filed on Mar. 30, 2023.

(51) Int. Cl. A61K 31/519; C07D 495/04; C07D 495/14

(52) U.S. Cl. CPC A61K 31/519; C07D 495/04; C07D 495/14

(57) ABSTRACT

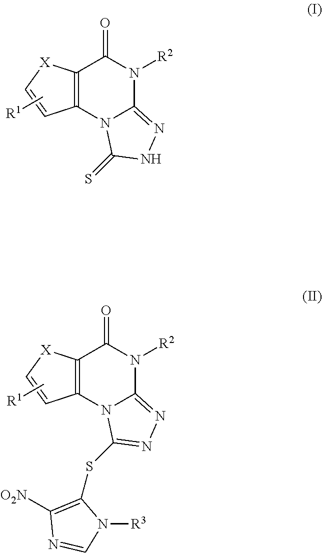

Disclosed are allosteric inhibitors of the Plk1 Polo Box Domain (PBD), which are useful in treating cancers by inducing mitotic block of the cancer cells. Examples of such inhibitors include compounds of formula (I) and (II), as well as pharmaceutically acceptable salt thereof, wherein X, R¹, R², and R³ are as defined. Also disclosed are pharmaceutical compositions containing a compound or salt thereof and a method of treating cancer in an animal by administering to the animal an effective amount of the compound or the pharmaceutical composition.

### CROSS-REFERENCE TO A RELATED APPLICATION

This patent application claims the benefit of U.S. Provisional Patent Application No. 63/455,608 filed Mar. 30, 2023, the disclosure of which is incorporated by reference in its entirety for all purposes.

### STATEMENT REGARDING FEDERALLY SPONSORED RESEARCH OR DEVELOPMENT

This invention was made with Government support under Intramural grant numbers ZIADK031117 and ZIABC010681. The Government has certain rights in the invention.

### BACKGROUND OF THE INVENTION

Members of the Polo subfamily of Ser/Thr protein kinases (collectively, polo-like kinases) play a key role in regulating various aspects of the cell cycle and cell proliferation (Zitouni et al., Nat. Rev. Mol. Cell Biol. 2014, 15 (7), 433-452). Among them, polo-like kinase 1 (Plk1) is critically required for proper mitotic progression, whereas other members play distinct roles during interphase progression and exhibit little functional overlap with other Plk family members (Zitouni et al., vide supra; and Lee et al., Trends Pharmacol. Sci. 2015, 36 (12), 858-877). In accordance with its importance in promoting mitosis, Plk1 is largely upregulated in a broad range of human cancers and its level of overexpression appears to correlate with aggressiveness and poor prognosis for a wide spectrum of human cancers (Strebhardt et al., Nat. Rev. Drug Discov. 2010, 9 (8), 643-660; and de Carcer et al., Genes (Basel) 2019, 10 (3), 208-221). Notably, various cancer cells, but not their isogenic normal cells, are addicted to high Plk1 levels and consequently require Plk1 overexpression for their viability (Luo et al., Cell 2009, 137 (5), 835-848; Sur et al., Proc. Natl. Acad. Sci. USA. 2009, 106 (10), 3964-3969; and Park et al., Cell Cycle 2015, 14 (22), 3624-3634). Since the reversal of addicted protein functions in cancer cells has proven to be an attractive strategy to selectively kill cancer cells (Weinstein et al., Science 2002, 297 (5578), 63-64; McMurray rt al., Nature 2008, 453 (7198), 1112-1126; and Luo et al., Cell 2009, 136 (5), 823-837), Plk1 is considered a discriminating target for anticancer therapy.

Plk1 contains an N-terminal kinase domain (KD) for ATP-dependent catalysis and is characterized by the presence of the C-terminal non-catalytic, but functionally essential, polo-box domain (PBD) (Elia et al., Cell 2003, 115 (1), 83-95; and Lee et al., Proc. Natl. Acad. Sci. USA 1998, 95 (16), 9301-9306). The PBD plays a key role in mediating Plk1 functions by targeting its N-terminal catalytic activity to distinct subcellular structures, such as centrosomes, kinetochores, and midbody, through specific protein-protein interactions (PPIs) (Lee et al., 1998, vide supra; and Seong et al., J. Biol. Chem. 2002, 277 (35), 32282-32293). For more than a decade, extensive efforts were made to develop Plk1 inhibitors targeting the KD, resulting in several Plk1 ATP-competitive inhibitors, such as Volasertib/BI6727 (Rudolph et al., Clin. Cancer Res. 2009, 15 (9), 3094-3102), BI2536 (Steegmaier et al., Curr. Biol. 2007, 17 (4), 316-322), GSK461364 (Russo et al., Thyroid 2013, 23 (10), 1284-1293), NMS-P937 (Beria et al., Bioorg. Med. Chem. Lett. 2011, 21 (10), 2969-2974), and TAK-960 (Nie et al., Bioorg. Med. Chem. Lett. 2013, 23 (12), 3662-3666) that have been examined against various cancers (Lee, 2015, vide supra). However, they all showed limited efficacy with more-than-acceptable dose-limiting toxicity in various preclinical or clinical trials. Since dose-limiting toxicity arises mainly from non-specific activity of these inhibitors (Karaman et al., Nat. Biotechnol. 2008, 26 (1), 127-132), improving Plk1 specificity is likely the major concern that needs to be addressed to achieve better clinical outcomes. In fact, one of the common problems associated with the currently available Plk1 ATP-competitive inhibitors appears to be their low degree of selectivity against other kinases (Lee et al., Trends Pharmacol. Sci. 2015, 36 (12), 858-877; and Park et al., F1000Res. 2017, 6, 1024), including two closely related Plk2 and Plk3 with potential tumor suppressor roles (Syed et al., Blood 2006, 107 (1), 250-256; and Yang et al., Cancer Res. 2008, 68 (11), 4077-4085).

Thus, there remains an unmet need for compounds that exhibit specific anti-Plk1 PBD activity for use in a method of treating cancer.

### BRIEF SUMMARY OF THE INVENTION

The invention provides compounds and prodrugs, as well as pharmaceutical compositions comprising such compounds and/or prodrugs, and a method for treating cancers, wherein the compounds and prodrugs act as allosteric inhibitors of the PLK 1 PBD of the cancerous cell or tissue.

### BRIEF DESCRIPTION OF THE SEVERAL VIEWS OF THE DRAWINGS

FIG. 1 depicts a method for synthesis of compounds suitable for making prodrugs in accordance with an aspect of the invention.

FIG. 2 depicts a method for synthesis of S-aryl prodrugs of active drugs shown in FIG. 1.

FIG. 3 depicts a method of synthesis of Biotin conjugate 22 in accordance with an aspect of the invention.

FIG. 4 illustrates the binding of Applopole-A to an allosteric site in accordance with an aspect of the invention.

FIG. 5 illustrates the inhibition by Allopole of PBD1-dependent interactions and subcellular localizations leading to mitotic block in Hela cells. FIG. 5a depicts the immunoprecipitation and immunoblotting analyses. FIG. 5b displays the results of confocal imaging and quantification of Plk1 signals. FIG. 5c displays the results of time-lapse analyses for HeLa cells released from a double thymidine (DT) block (G1/S) and treated with DMSO or Allopole 7 h after release.

FIG. 6a depicts a model showing Allopole-A sandwiched between W410 and F559 residues of PBD1. FIG. 6b depicts an overlay of PBD1.Allopole-A and PBD2. FIG. 6c depicts an overlay of PBD1.Allopole-A and PBD3.

FIG. 7 shows that mutation of F559 to a charged residue renders Plk1 insensitive to Allopole treatment in vitro and in vivo. FIG. 7(a) shows the results of FP-based assays showing that a FP-based assays showing the binding of FITC-Ahx-DPPLHS-pT-AI-NH2 {Qian, 2014#1416} to the different concentrations of WT or mutant forms of PBD1. Bars, mean #s.d. FIG. 7(b) depicts the results of FP-based inhibition assays for the interaction between FITC-Ahx-DPPLHS-pT-AI-NH2 and the indicated PBD1 WT or mutants in the presence of various concentrations of Allopole. Bars, mean±s.d. FIG. 7(c) depicts confocal imaging and quantification of Plk1 endogenous promoter (Pendo)-fused mGFP-Plk1 signals in U2OS cells silenced for endogenous Plk1 (siPlk1) and treated with 8 μM of Allopole for the indicated length of time (Experimental procedure at bottom, right). Arrowheads, centrosomes. Quantification of Pendo-mGFP-Plk1 signals was performed with the images acquired from three independent experiments [n=a total of 93, 53, and 79 cells for Plk1 WT samples (in order) and n=a total of 92, 64, and 76 cells for the F559D mutant samples (in order)]. Bars, mean±s.d. Arrowheads, centrosomes. ****, P<0.0001 (unpaired two-tailed t-test).

FIG. 8 depicts the activities of various compounds in accordance with an aspect of the invention.

FIG. 9 depicts the results of PBD1 binding assay in accordance with an aspect of the invention.

FIG. 10A depicts the structural formulas for Allopole-A, Allopole, and prodrugs NCK197 to NCK199. FIG. 10B depicts the structural formula of NCK200.

FIG. 11A depicts the test results obtained from in vitro ELISA-based PBD inhibition assay (B) and colorimetric MTS-based anti-cell proliferation assay (C). FIG. 11B depicts the results of the MTS assay for NCK190, NCK198, and NCK200 and of the ELISA assay for NCK189 and NCK198.

FIG. 12 depicts a list of additional compounds in an aspect of the invention.

FIG. 13 depicts results obtained on certain prodrugs from tests based on in vitro-fluorescence polarization-based PBD inhition assay in an aspect of the invention.

FIG. 14 depicts the PK profiles of (A) NCK182 and its metabolites, (B) NCK181, and (C) glucuronidated NCK1 in mice following i.p. injection of NCK182 at 15 mg/kg, in an aspect of the invention.

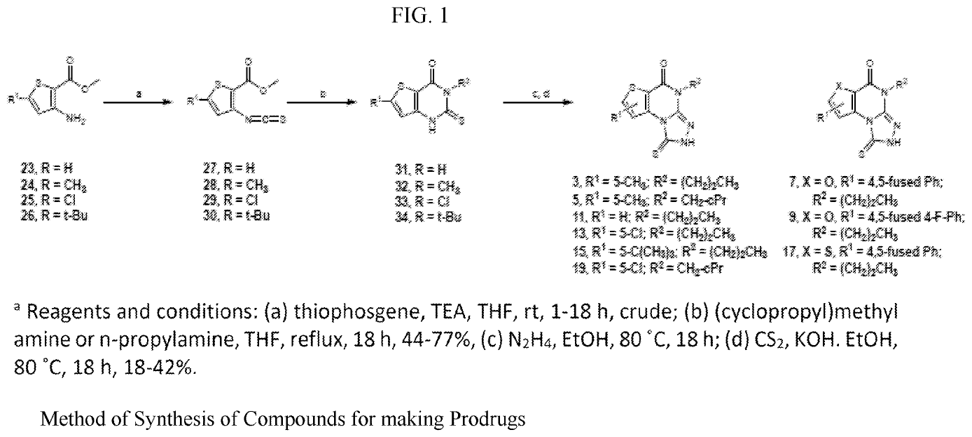

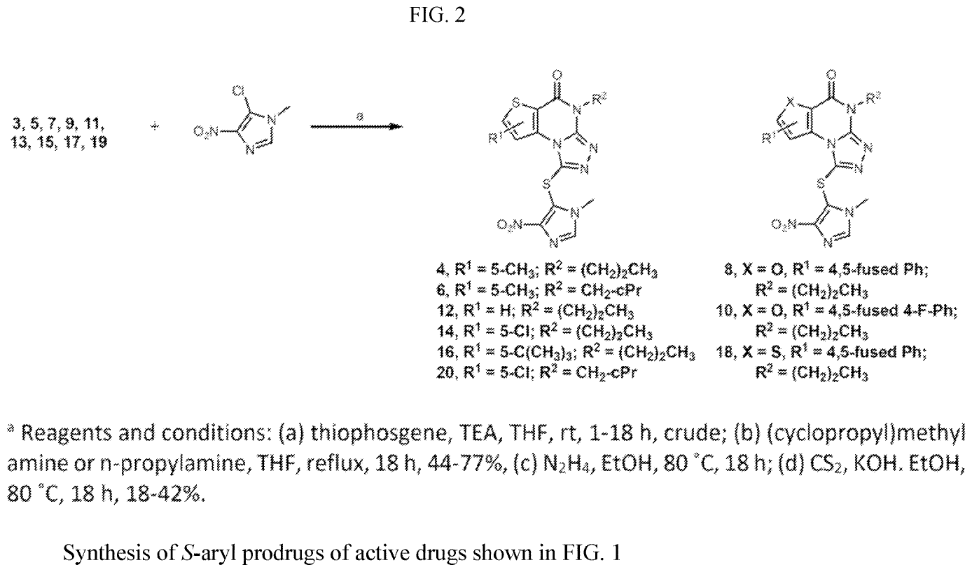

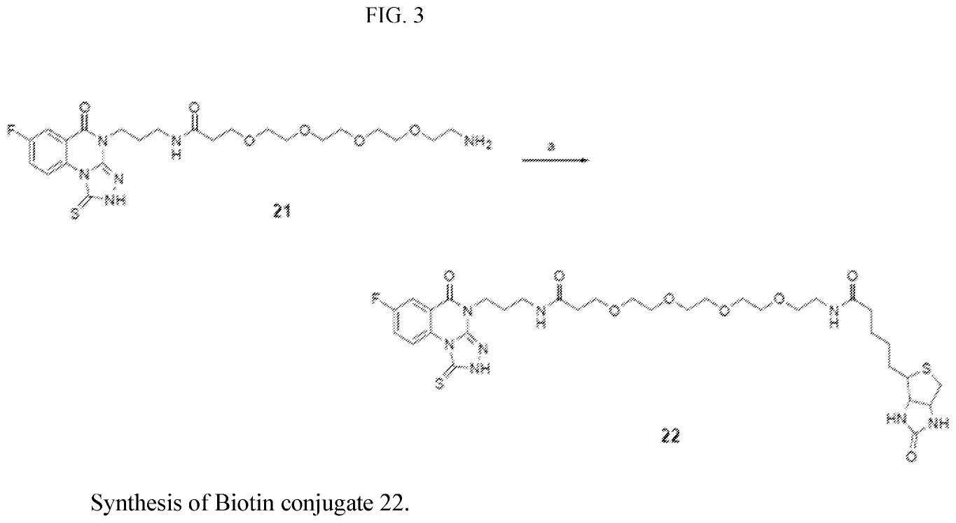

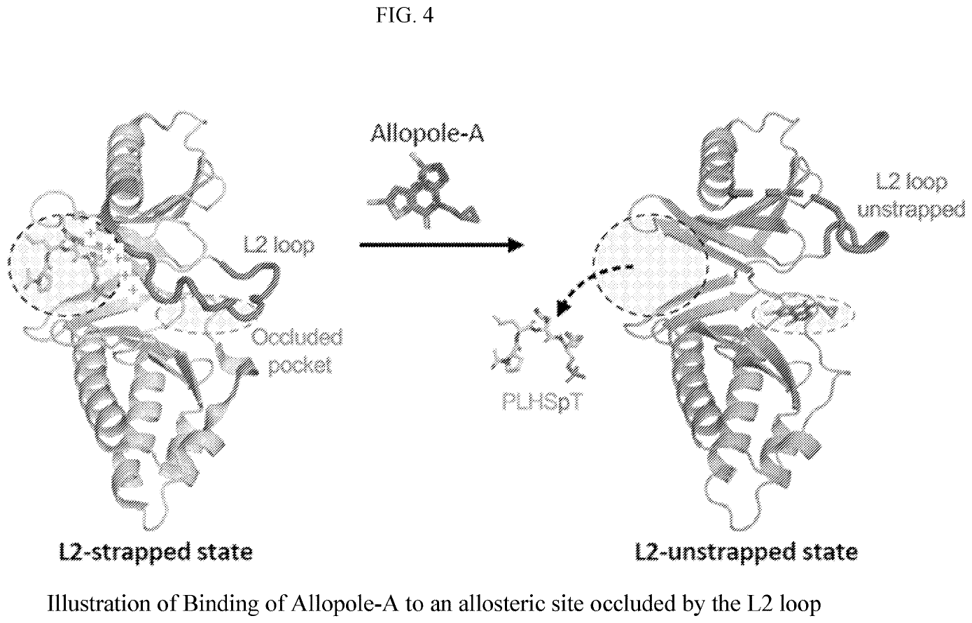

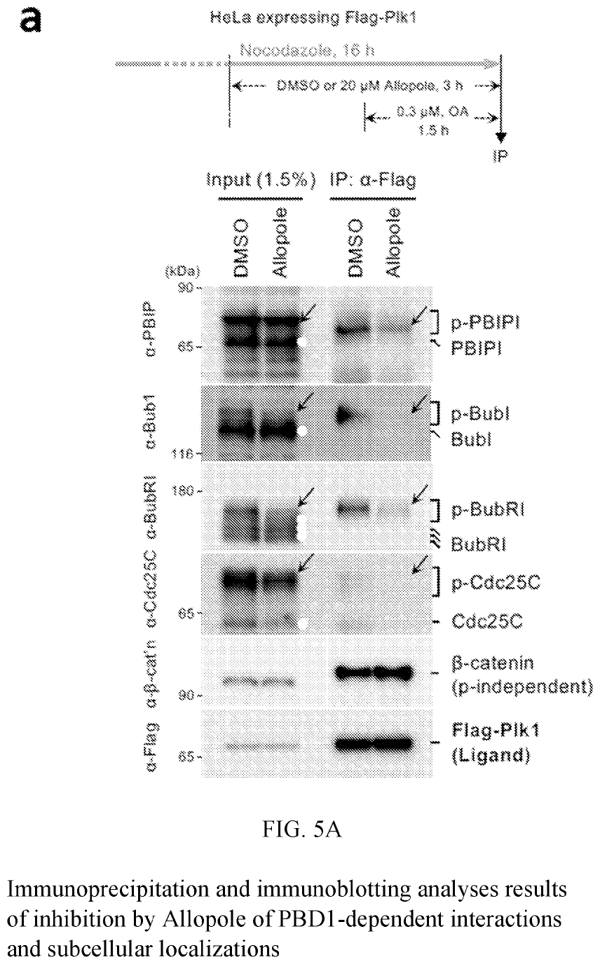

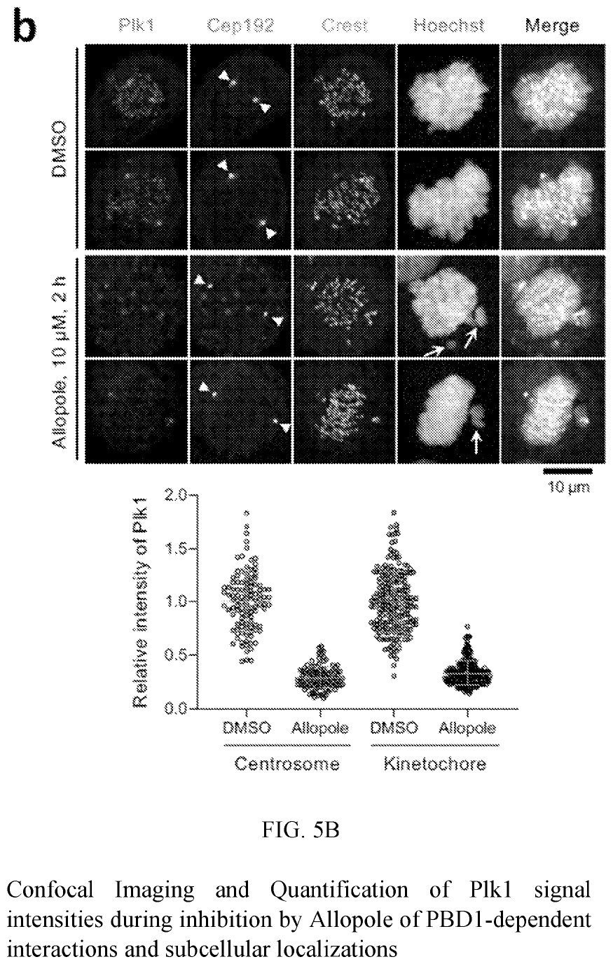

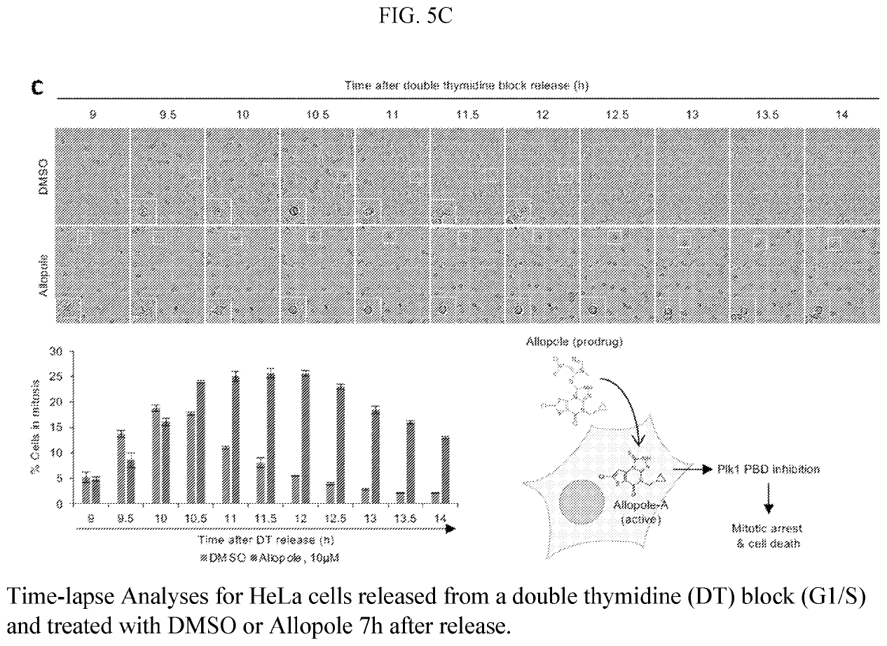

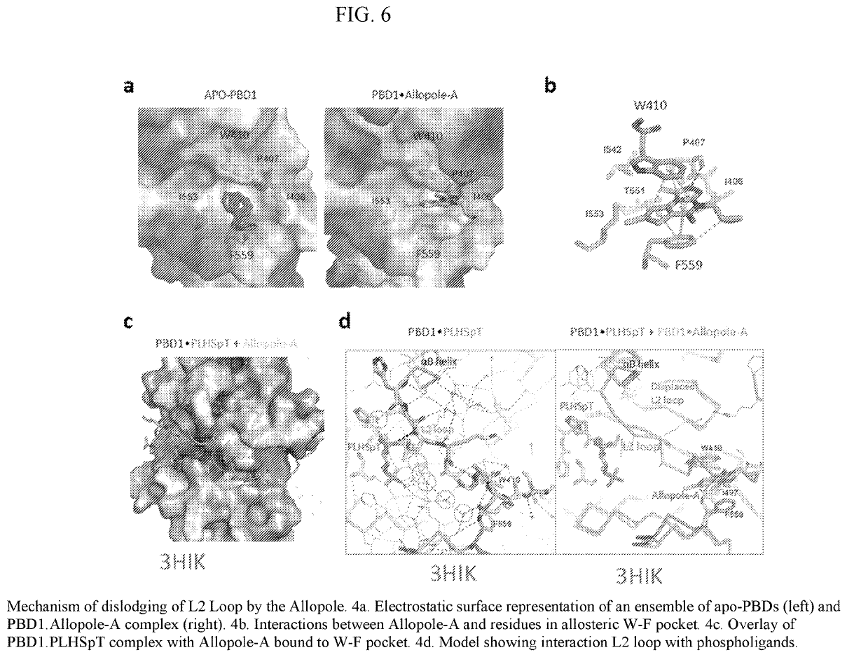

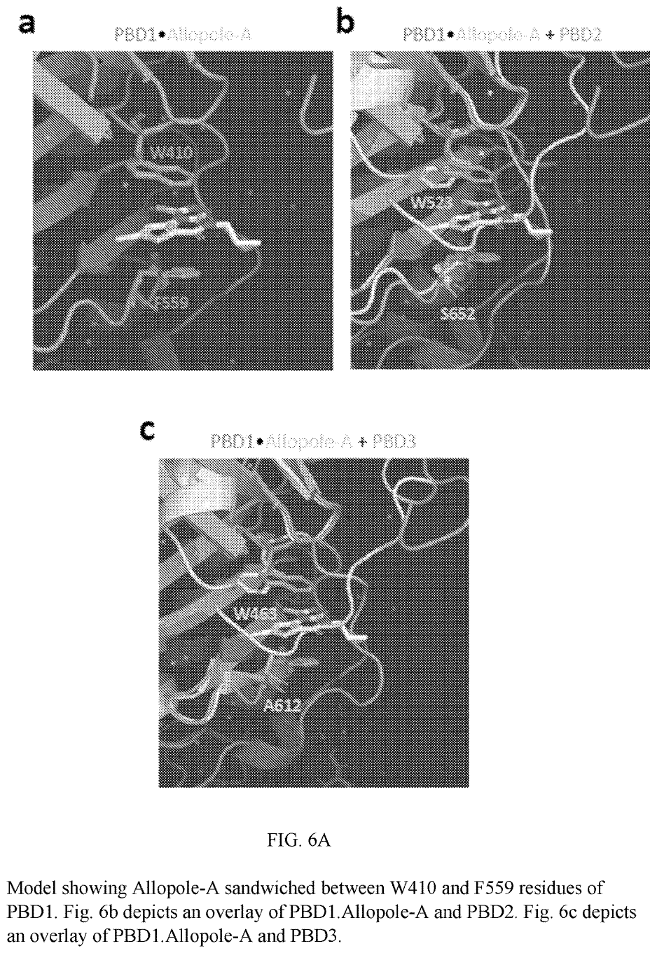

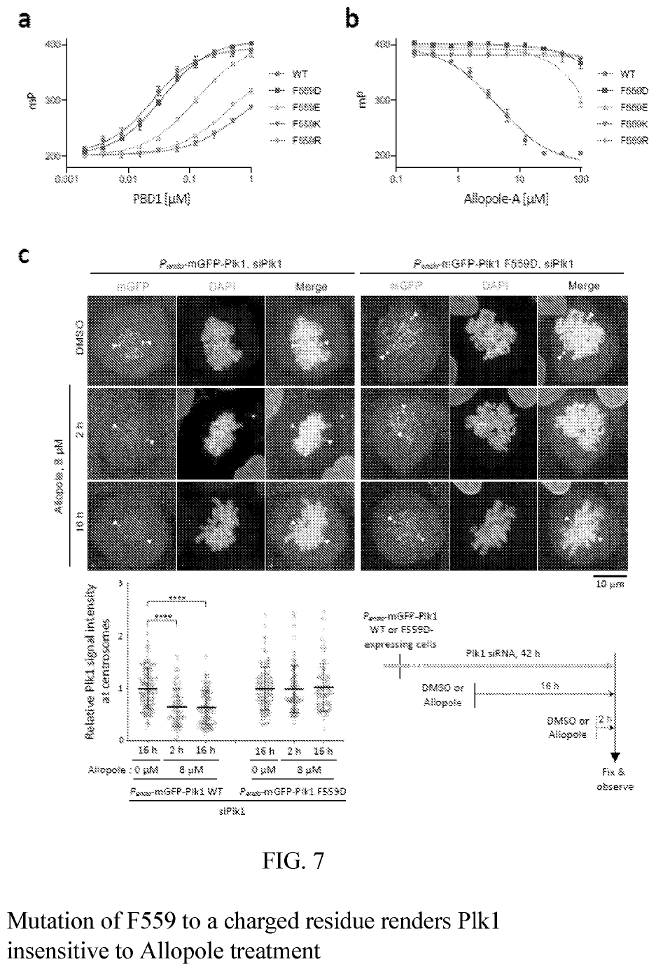

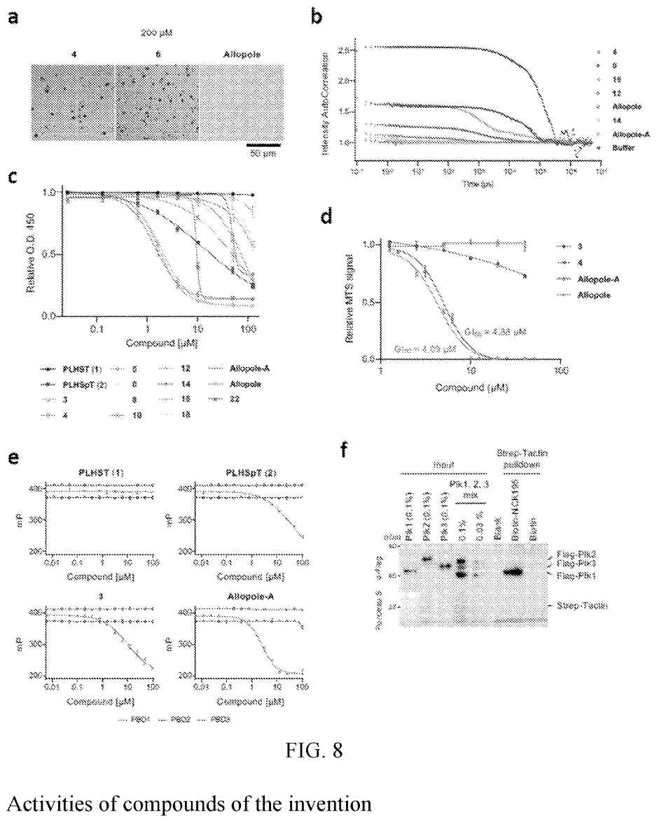

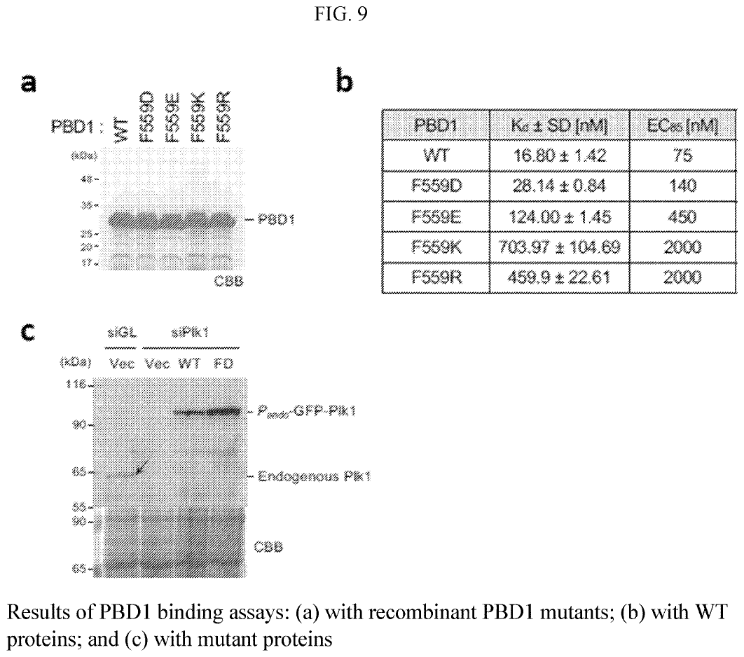

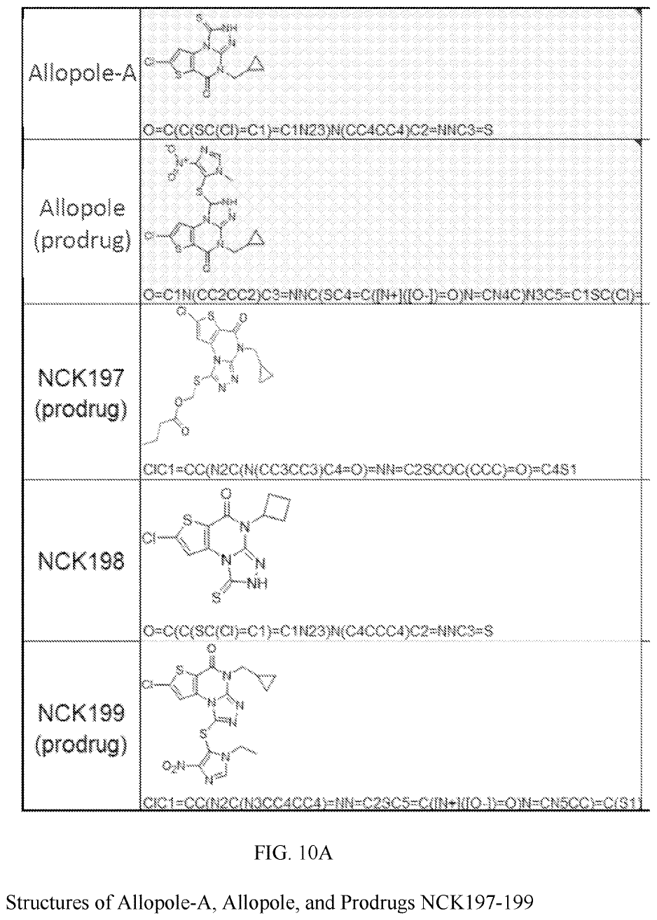

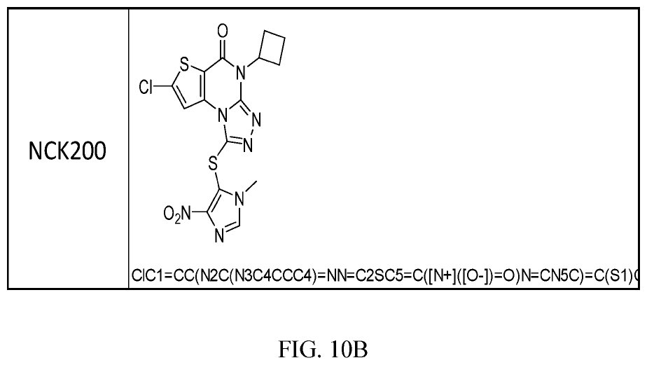

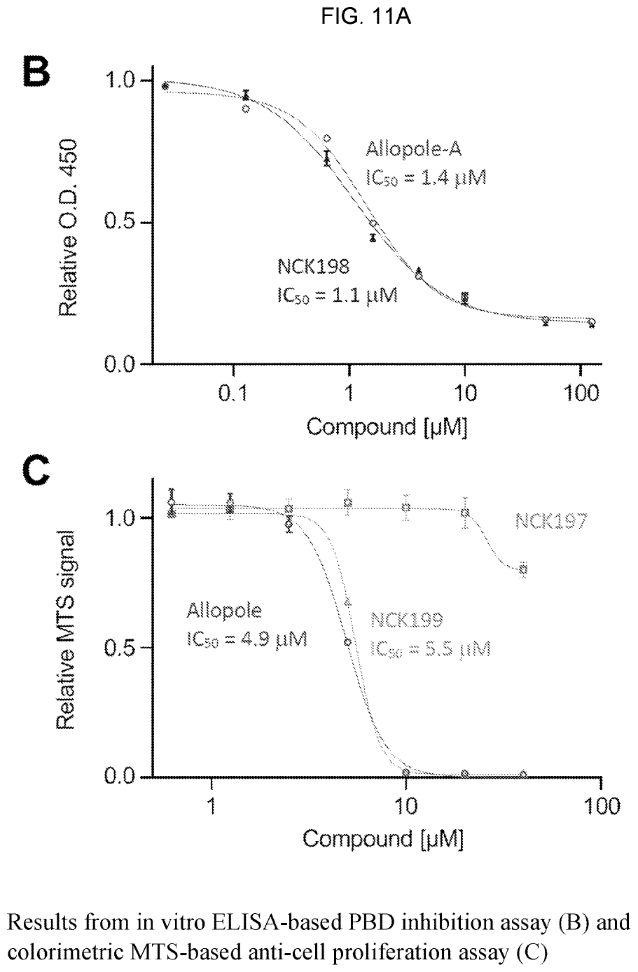

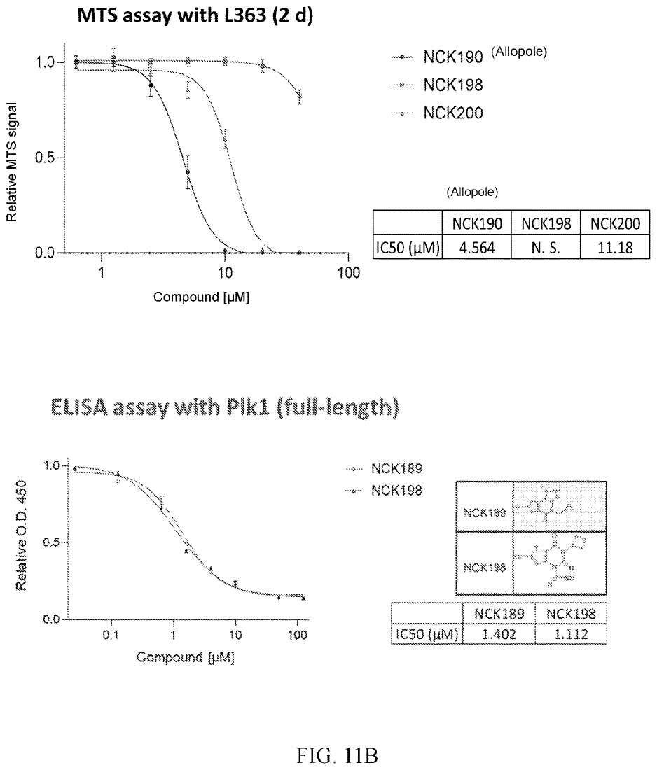

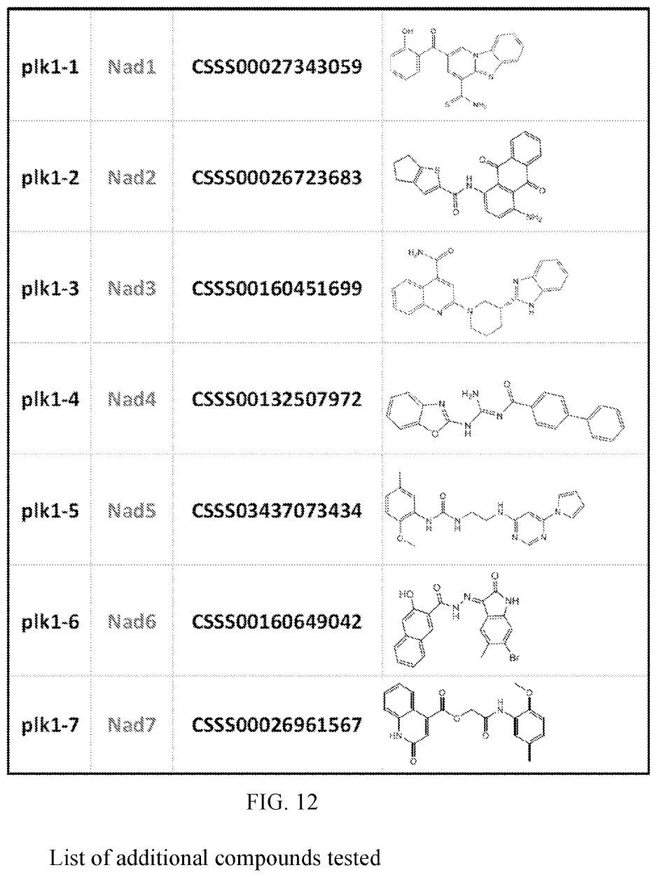

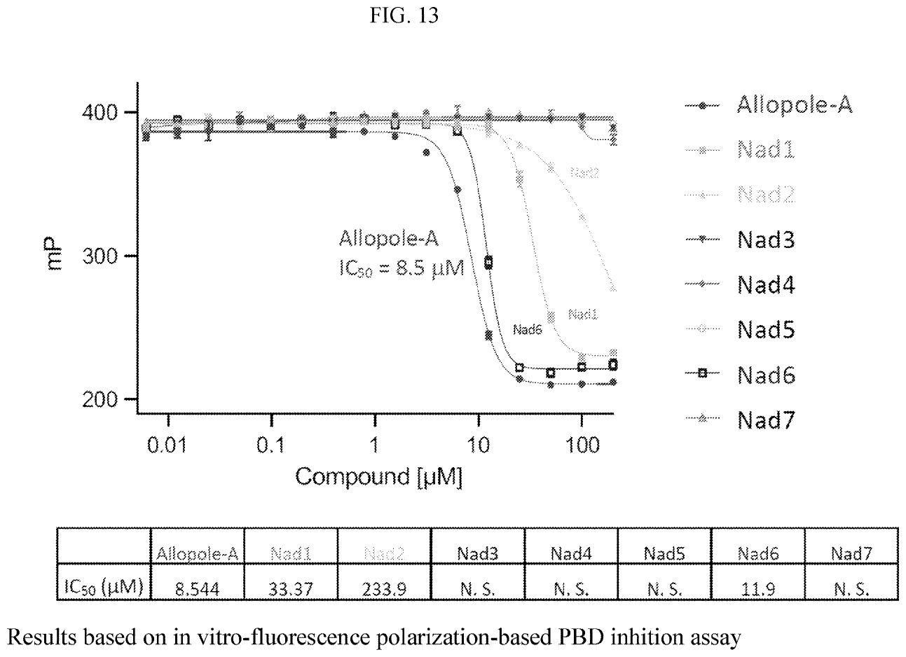

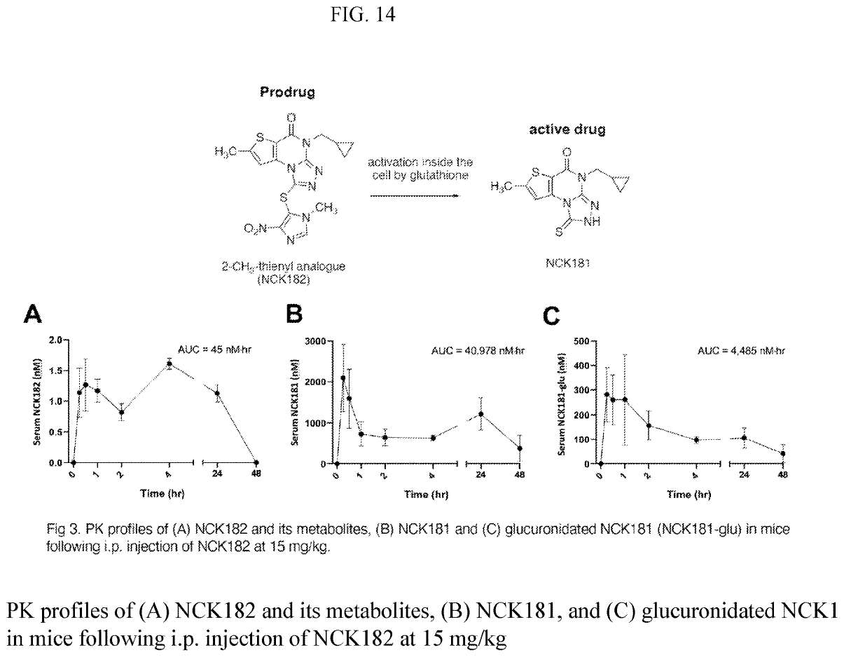

### DETAILED DESCRIPTION OF THE INVENTION

In an aspect, the invention provides a compound of formula (I) or (II),

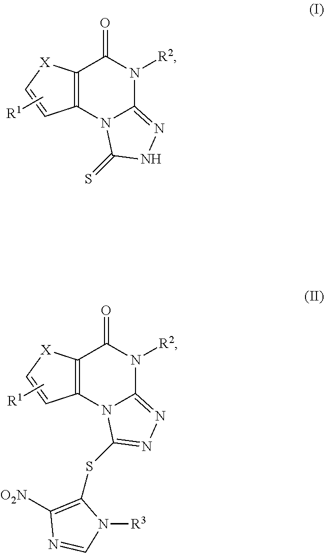

wherein X is O or S;
R¹ is H, halo, C₁-C₆ alkyl, C₃-C₈ cycloalkyl, C₃-C₈ cycloalkyl C₁-C₃ alkyl, 4,5-fused phenyl, or 4,5-fused halophenyl;
R² is halo or C₁-C₆ alkyl; and
R³ is C₁-C₃ alkyl;
or a compound selected from the group consisting of compounds Nad-1 to Nad-8 and CIL-1 to CIL-18 of the formulas:

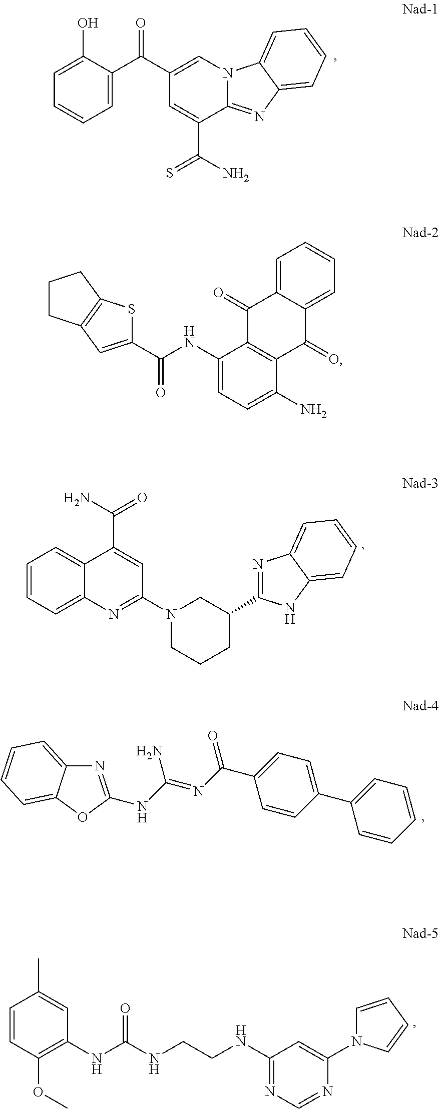 [SMILES: NC(=S)c1cc(C(=O)c2ccccc2O)cn2c1nc1ccccc12 ; Nc1ccc(NC(=O)c2cc3c(s2)CCC3)c2c1C(=O)c1ccccc1C2=O ; NC(=O)c1cc(N2CCC[C@@H](c3nc4ccccc4[nH]3)C2)nc2ccccc12 ; N/C(=N\C(=O)c1ccc(-c2ccccc2)cc1)Nc1nc2ccccc2o1 ; COc1ccc(C)cc1NC(=O)NCCNc1cc(-n2cccc2)ncn1]

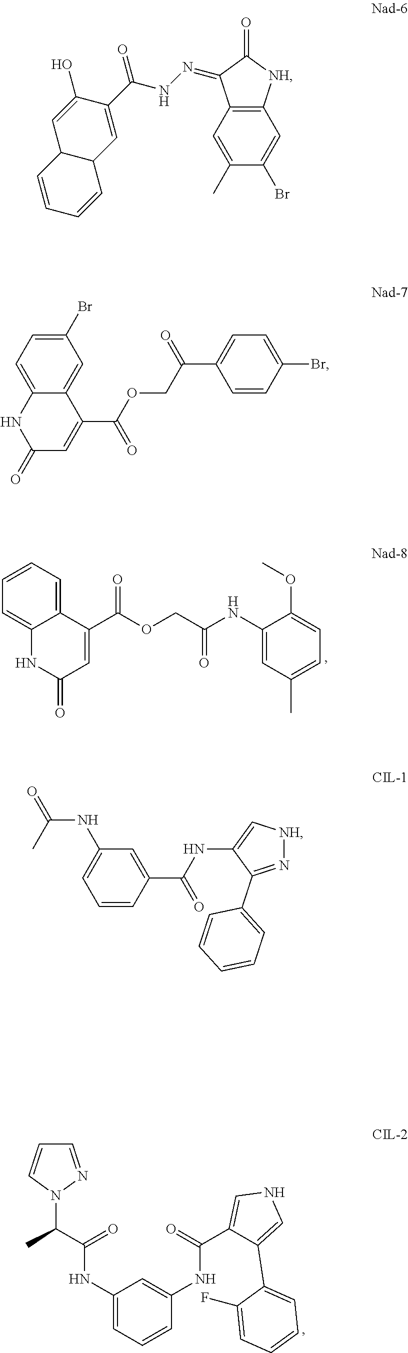 [SMILES: Cc1cc2c(cc1Br)NC(=O)/C2=N/NC(=O)C1=CC2C=CC=CC2C=C1O ; O=C(COC(=O)c1cc(=O)[nH]c2ccc(Br)cc12)c1ccc(Br)cc1 ; COc1ccc(C)cc1NC(=O)COC(=O)c1cc(=O)[nH]c2ccccc12 ; CC(=O)Nc1cccc(C(=O)Nc2c[nH]nc2-c2ccccc2)c1 ; C[C@H](C(=O)Nc1cccc(NC(=O)c2c[nH]cc2-c2ccccc2F)c1)n1cccn1]

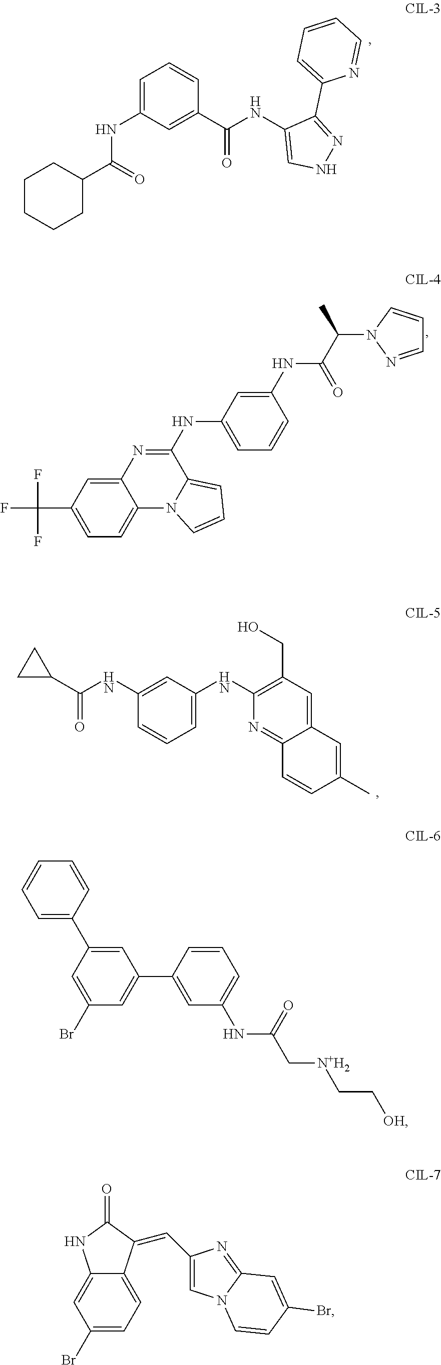

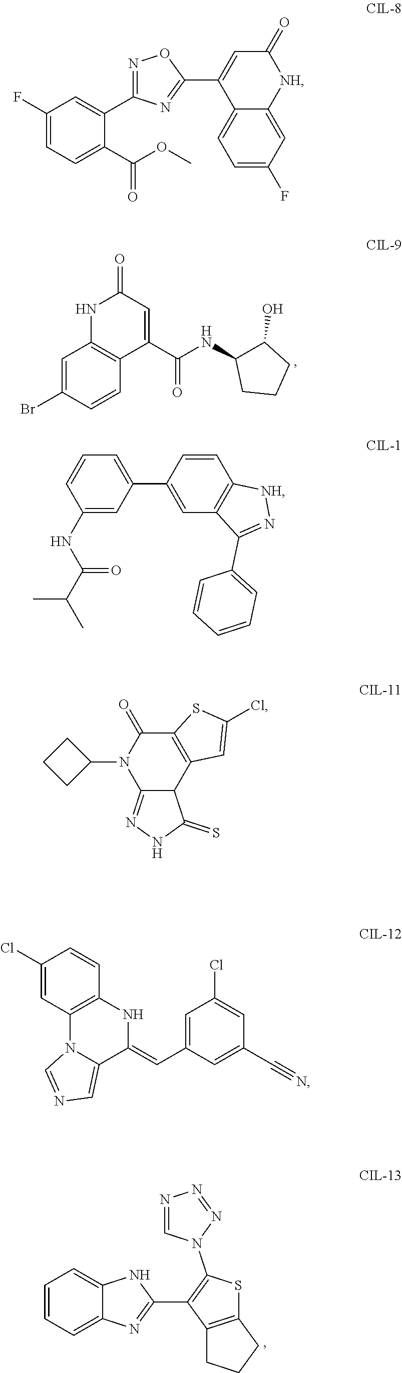 [SMILES: COC(=O)c1ccc(F)cc1-c1noc(-c2cc(=O)[nH]c3cc(F)ccc23)n1 ; O=C(N[C@@H]1CCC[C@H]1O)c1cc(=O)[nH]c2cc(Br)ccc12 ; CC(C)C(=O)Nc1cccc(-c2ccc3[nH]nc(-c4ccccc4)c3c2)c1 ; O=C1c2sc(Cl)cc2C2C(=S)NN=C2N1C1CCC1 ; N#Cc1cc(Cl)cc(/C=C2\Nc3ccc(Cl)cc3-n3cncc32)c1 ; c1ccc2[nH]c(-c3c(-n4cnnn4)sc4c3CCC4)nc2c1]

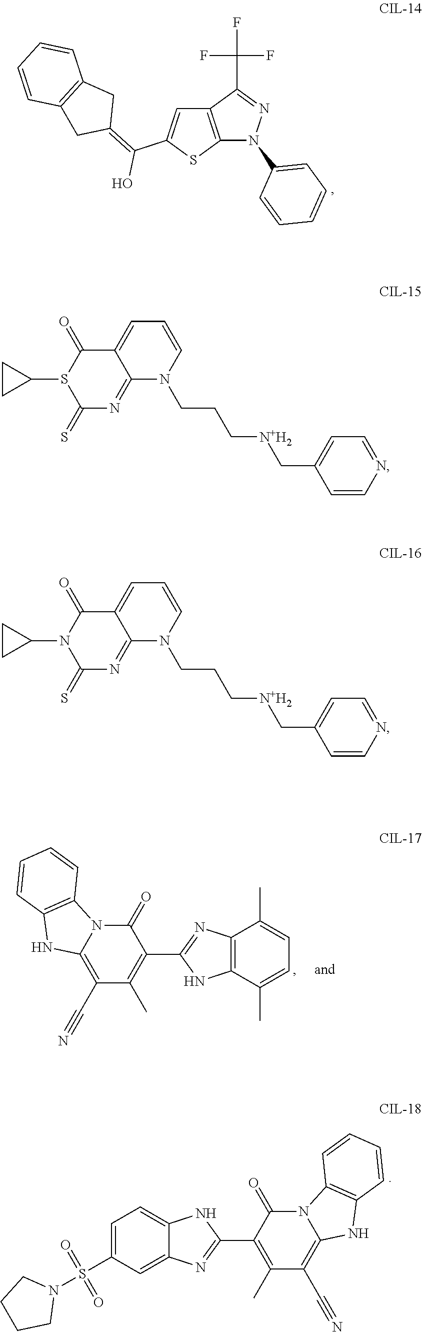

In an aspect, the present invention provides a compound of formula 22, and compounds of formula (A), (B), (C), and (D), and pharmaceutically acceptable salts thereof:

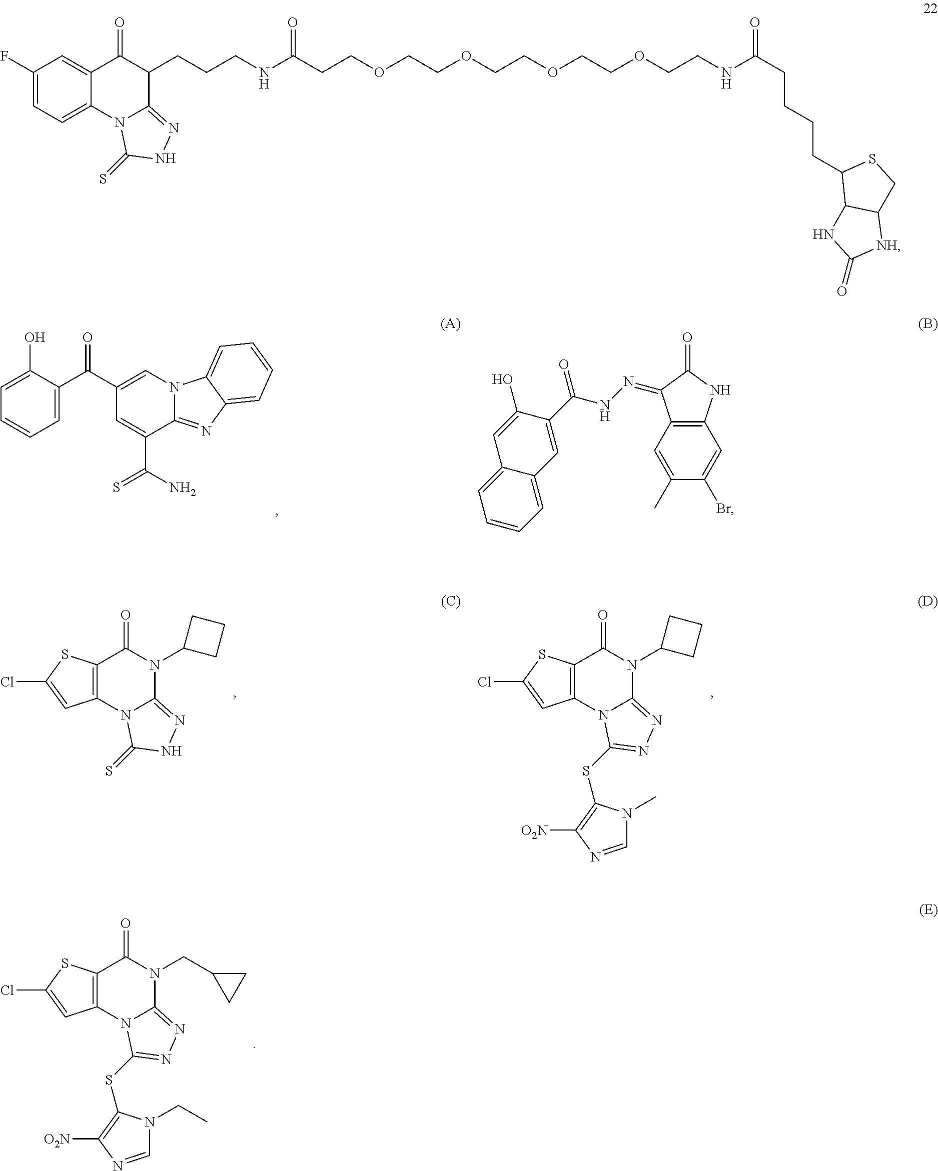 [SMILES: O=C(CCCCC1SCC2NC(=O)NC21)NCCOCCOCCOCCOCCC(=O)NCCCC1C(=O)c2cc(F)ccc2-n2c1n[nH]c2=S ; NC(=S)c1cc(C(=O)c2ccccc2O)cn2c1nc1ccccc12 ; Cc1cc2c(cc1Br)NC(=O)/C2=N/NC(=O)c1cc2ccccc2cc1O ; O=c1c2sc(Cl)cc2n2c(=S)[nH]nc2n1C1CCC1 ; Cn1cnc([N+](=O)[O-])c1Sc1nnc2n(C3CCC3)c(=O)c3sc(Cl)cc3n12 ; CCn1cnc([N+](=O)[O-])c1Sc1nnc2n(CC3CC3)c(=O)c3sc(Cl)cc3n12]

In some aspects, the following compounds of formula (I) can be used in a method of treating Plk1-mediated cancer in a subject in need thereof (e.g., a subject with cancer cells that overexpress Plk1 relative to normal tissue of the same type). The method comprises administering to the subject in need one or more compounds as described above.

The compounds and prodrugs of the invention can be made by any suitable method, for example, those set forth in the Examples.

In any of the aspects herein, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

In any of the aspects herein, the term “prodrug” is intended to include any compound that releases an active parent drug according to a structure described herein in vivo when such prodrug is administered to a subject. Prodrugs of a compound described herein are prepared by modifying functional groups present in the compound described herein in such a way that the modifications can be cleaved in vivo to release the active parent compound.

The pharmaceutically acceptable carriers are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intraperitoneal, intramuscular, intratumoral, peritumoral, intraperitoneal, intrathecal, rectal, vaginal, or aerosol formulations. In some aspects, the pharmaceutical composition is administered orally or intravenously.

In accordance with any of the aspects, any of the compounds of the invention or a pharmaceutically acceptable salt thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I) or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the inhibitors in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The inhibitors may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The compound or a pharmaceutically acceptable salt thereof, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular inhibitor and the desired effect. It will be appreciated by one of skill in the art that various conditions or disease states may require prolonged treatment involving multiple administrations.

The inventive methods comprise administering an effective amount of a compound of the invention or a pharmaceutically acceptable salt thereof. An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular cancer. The meaningful benefit observed in the subject can be to any suitable degree (10, 20, 30, 40, 50, 60, 70, 80, 90% or more). In some aspects, one or more symptoms of the cancer are prevented, reduced, halted, or eliminated subsequent to administration of a compound or a pharmaceutically acceptable salt thereof, thereby effectively treating the cancer to at least some degree.

Effective amounts may vary depending upon the biological effect desired in the individual, condition to be treated, and/or the specific characteristics of the compound of the invention or a pharmaceutically acceptable salt thereof, and the individual. In this respect, any suitable dose of the compound of the invention or a pharmaceutically acceptable salt thereof can be administered to the subject (e.g., human), according to the type of cancer to be treated. Various general considerations taken into account in determining the “effective amount” are known to those of skill in the art and are described, e.g., in Gilman et al., eds., Goodman and Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound or a pharmaceutically acceptable salt thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

In an aspect, compounds of the invention inhibit Plk1, particularly the PBD of Plk1. For example, the compounds can be at least 2 times (e.g., at least 3 times, at least 4 times, at least 5 times, at least 6 time, at least 8 times, at least 10 times, at least 15 times, at least 20 times, at least 30 times, at least 40 times, at least 50 times, at least 60 times, or at least 100 times) more selective for Plk1 compared to one or more other polo-like kinases.

Since PBD inhibitors interfere only with the PBD-dependent Plk1 functions, they are anticipated to incur mitotic stress sufficient to induce cell death in cancer cells but not in normal cells. See, e.g., Park et al., Cell Cycle 2015, 14 (22), 3624-3634. While not wishing to be bound by any particular theory, it is believed that complete inhibition of Plk1 would be detrimental even for normal cell proliferation. Thus, inhibition of the PBD of Plk1 is seen as a viable treatment of cancer. Anti-cancer activity can be measured by any suitable method, including the assays described herein.

The type of cancer is not particularly limited, but in certain aspects, the cancer comprises cancer cells that overexpress Plk1 relative to normal tissue of the same type. Examples of cancer treatable with the inventive method include cancers, including cancerous cells and tissue, of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-borne tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocytic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th Ed. 2001). In some aspects, the cancer is breast cancer, lung cancer, renal cancer, liver cancer, uterine cancer, prostate cancer, pancreatic cancer, glioma, thyroid carcinoma, head and neck squamous cell carcinoma, melanoma, colorectal cancer, esophageal carcinoma, or ovarian carcinoma.

In certain aspects of this method, the compound of the invention or a pharmaceutically acceptable salt thereof can be co-administered with an anti-cancer agent (e.g., a chemotherapeutic agent) and/or radiation therapy. In an aspect, the method comprises administering an amount of a compound of the invention or a pharmaceutically acceptable salt thereof that is effective to sensitize the cancer cells to one or more therapeutic regimens (e.g., chemotherapy or radiation therapy). The terms “co-administered” or “co-administration” refer to simultaneous or sequential administration. A compound of the invention or a pharmaceutically acceptable salt thereof can be administered before, concurrently with, or after administration of another anti-cancer agent (e.g., a chemotherapeutic agent).

One or more than one, e.g., two, three, or more anti-cancer agents can be administered. In this regard, the present invention is directed a pharmaceutical composition comprising a pharmaceutically acceptable carrier and a combination of the compound of the invention or a pharmaceutically acceptable salt thereof and at least one anti-cancer agent (e.g., chemotherapeutic agent).

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mitomycin C, plicamycin, dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panitumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, cellular immunotherapy (e.g., chimeric antigen receptor T cell therapy, tumor-infiltrating lymphocyte therapy), or any combination thereof. In some aspects, the anti-cancer agent is cisplatin, cytarabine, methotrexate, doxorubicin, or a combination thereof. In other aspects, the anti-cancer agent is imatinib, idelalisib, lapatinib, dasatinib, ceritinib, crizotinib, or a combination thereof.

In certain aspects of the method, the compound of the invention or a pharmaceutically acceptable salt thereof can be co-administered with one or more antifungal agents, particularly, an antifungal agent that inhibits CYP 3A4, such as ketoconazole, fluconazole, itraconazole, miconazole, posaconazole, voriconizole, or ritonavir. In some preferred aspects, the antifungal agent is ketoconazole.

One or more than one, e.g., two, three, or more antifungal agents can be administered. In this regard, the present invention is directed a pharmaceutical composition comprising a pharmaceutically acceptable carrier and a combination of the compound of the invention or a pharmaceutically acceptable salt thereof and at least one antifungal agent.

The invention is further illustrated by the following examples.

### Example 1

Chemical synthesis of Allopole and its related compounds was carried out as follows. The reactions were performed under anhydrous conditions under dry nitrogen. For particularly sensitive reactions, the glassware was oven-dried. Reactants used were reagent grade, and anhydrous solvents were purchased commercially. Compounds 3, 4, 7, 9, 11, 13, 15, 17 and 21 were synthesized in a manner similar to that was reported in {Park, 2023 #3387} and {Alverez, 2020 #3186}. Room temperature is defined as 25±2° C. All of the final products for biological testing were purified using semi-preparative HPLC (Waters, Beverly, MA), using a Phenomenex Luna C18 (5 μm, 30×75 mm) column with a 45 mL/min flow rate. The mobile phase gradient (10% to 50% acetonitrile over 8 min) consisted of acetonitrile/water mixtures, with TFA (0.1%) present in all mixtures. The course of the HPLC purification was followed by UV at 220 nm. Selected compounds were initially purified following dry-loading on an ISCO CombiFlash System (Teledyne, Lincoln, NE) prior to HPLC. Fractions obtained during the synthesis were examined for expected product using an Agilent 1200 LC-MS (Agilent Technologies, Santa Clara, CA) with a rapid gradient consisting of 4% to 100% CH₃CN in water over 3 min, with a total separation time of 8 min (1 mL/min). The CH₃CN component contained 0.025% TFA, and the aqueous component contained 0.05% TFA. The purity of the final products (>95% at 254 nm) was shown using the Agilent 1200 LC-MS with a sequential 7-min linear gradient of 4% to 100% acetonitrile (with TFA, column and flow rate, as above) and a subsequent 4.5-min run at 50° C. The purity of the commercial starting materials was confirmed to be >95% (254 nm) using an ZORBAX Eclipse XDB C₁₈ column (5 mm, 4.6×250 mm, Agilent Technologies) with a linear gradient of 5% to 95% acetonitrile in water (containing 10 mM triethylammonium acetate) for 20 min at a flow rate of 1.0 mL/min. 1H and ¹³C NMR spectra were measured using either a Varian 400 (100) MHz or a Bruker 400 MHz instrument. Chemical shifts in ppm were relative to the small proton signals from solvent. For 19F NMR (BrukerTopspin/MestReNova 10.0.2 or 14.1.0), chemical shifts were solvent-calibrated internally. High resolution mass spectrometry (HRMS) was recorded using either 6210 Time-of-Flight LCMS system (Agilent Technologies) or a Micromass spectrometer (Waters) equipped with a standard interface (electrospray ionization (ESI) and modular LockSpray™).
General Procedure A: A solution of methyl ester 23-26 (1.0 mmol) in THF (5 mL) was treated with triethylamine (3.0 mmol) and cooled to OC in an ice bath and followed by thiophosgene (1.1 mmol). The reaction mixture was slowly warmed to room temperature and stirred for 1 h to overnight. Water was added to quench the reaction, and the mixture was extracted with ether. The organic layer was dried with MgSO₄ and filtered, and the filtrate was concentrated. The reaction mixture containing isothiocyanate 27-30 was used for the next reaction without further purification.
General Procedure B: To crude isothiocyanate 27-30 (1.0 mmol) in THF (1.5 mL) was added (cyclopropyl)methylamine (1.1 mmol), and the mixture was refluxed for 18 h. After cooling and dilution with water, the mixture was extracted with ethyl acetate, the organic layer dried with MgSO₄ and filtered, and the filtrate was concentrated. The residue (compounds 31-34) was purified by column chromatography.
General Procedure C: Compounds 31-34: The compound (1.0 mmol) was dissolved in ethanol (3.0 mL), and anhydrous hydrazine (7.0 mmol) was added. The reaction mixture was heated to 80° C. for 18 h. The reaction mixture was cooled to room temperature and volume reduced under a stream of nitrogen. Ethanol (3.0 mL), potassium hydroxide (3.0 mmol) and carbon disulfide (3.0 mmol) were added, and the reaction mixture was heated to 80° C. for 18 h. The reaction mixture was cooled and treated dropwise with 1 N HCl to pH 1 with vigorous stirring, and the mixture extracted with ethyl acetate. The organic layer was dried with MgSO₄ and filtered. The filtrate was concentrated. The products (compounds 3, 5, 11, 13, 15 and 19) were isolated from the reaction mixture by column chromatography.
General Procedure D: A mixture of the active drug (compounds 3, 5, 7, 9, 11, 13, 15, 17 and 19) (1.0 mmol), water (3.2 mL) and sodium hydroxide (1.1 mmol) was stirred until a solution formed. The corresponding-1-methyl-4-nitroimidazole (1.0 mmol) was added to the reaction mixture and the mixture stirred for 3 h at room temperature. After completion, the suspension was neutralized with acetic acid and a solid precipitated. The precipitate was isolated by filtration and the solid recrystallized from ethanol. The product was purified by column chromatography.

4-(Cyclopropylmethyl)-7-methyl-1-thioxo-2,4-dihydrothieno[2,3-e][1,2,4]triazolo[4,3-a]pyrimidin-5 (1H)-one, 5: Methyl 3-isothiocyanato-5-methylthiophene-2-carboxylate 5 was synthesized according to General Procedure A using methyl 3-amino-5-methylthiophene-2-carboxylate, 24 as starting material. Compound 28 was converted to 3-(cyclopropylmethyl)-6-methyl-2-thioxo-2,3-dihydrothieno[3,2-d]pyrimidin-4 (1H)-one 32 according to General Procedure B. Compound 32 was converted to 4-(cyclopropylmethyl)-7-methyl-1-thioxo-2,4-dihydrothieno[2,3-e][1,2,4]triazolo[4,3-a]pyrimidin-5 (1H)-one 5 according to General Procedure C (42%). ¹H NMR (400 MHz, DMSO-d₆): δ 14.09 (s, 1H), 8.68 (s, 1H), 3.90 (t, J=7.1 Hz, 2H), 2.67 (s, 3H), 1.27 (m, 1H), 0.41-0.49 (m, 4H); HRMS m/z (M+H) for C₁₂H₁₃N₄OS₂ calculated 293.0531, found 293.0527.

4-(Cyclopropylmethyl)-7-methyl-1-((1-methyl-4-nitro-1H-imidazol-5-yl)thio) thieno[2,3-e][1,2,4]triazolo[4,3-a]pyrimidin-5 (4H)-one, 6:4-(Cyclopropylmethyl)-7-methyl-1-((1-methyl-4-nitro-1H-imidazol-5-yl)thio) thieno[2,3-e][1,2,4]triazolo[4,3-a]pyrimidin-5 (4H)-one, 6 was synthesized according to General Procedure D using 4-(cyclopropylmethyl)-7-methyl-1-thioxo-2,4-dihydrothieno[2,3-e][1,2,4]triazolo[4,3-a]pyrimidin-5 (1H)-one (5) as starting material (74%). ¹H NMR (400 MHz, DMSO-d₆): δ 8.13 (s, 1H), 7.97 (s, 1H), 4.05 (d, J=7.2 Hz, 2H), 3.85 (s, 3H), 2.70 (s, 3H), 1.32 (m, 1H), 0.46 (m, 4H); HRMS m/z (M+H) for C₁₆H₁₆N₇O₃S₂ calculated 418.0756, found 418.0757.

1-((1-Methyl-4-nitro-1H-imidazol-5-yl)thio)-4-propylbenzofuro[2,3-e][1,2,4]triazolo[4,3-a]pyrimidin-5(4H)-one, 8. 1-((1-Methyl-4-nitro-1H-imidazol-5-yl)thio)-4-propylbenzofuro[2,3-e][1,2,4]triazolo[4,3-a]pyrimidin-5 (4H)-one was synthesized according to General Procedure D using 4-propyl-1-thioxo-1,2-dihydrobenzofuro[2,3-e][1,2,4]triazolo[4,3-a]pyrimidin-5 (4H)-one (7, compound 40 in {Park, 2023 #3387}) as starting material (27%). ¹H NMR (400 MHz, DMSO-d₆): δ 9.15 (d, J=8.3 Hz, 1H), 8.15 (s, 1H), 8.00 (d, J=8.5 Hz, 1H), 7.78 (t, J=7.4 Hz, 1H), 7.60 (t, J=7.3 Hz, 1H), 4.21 (t, J=6.4 Hz, 2H), 3.88 (s, 3H), 1.79 (m, 1H), 0.93 (t, J=7.4 Hz, 3H); HRMS m/z (M+H) for C₁₈H₁₆N₇O₄S calculated 426.0984, found 426.0978.

10-Fluoro-1-((1-methyl-4-nitro-1H-imidazol-5-yl)thio)-4-propylbenzofuro[2,3-e][1,2,4]triazolo[4,3-a]pyrimidin-5 (4H)-one, 10. 10-fluoro-1-((1-methyl-4-nitro-1H-imidazol-5-yl)thio)-4-propylbenzofuro[2,3-e][1,2,4]triazolo[4,3-a]pyrimidin-5 (4H)-one was synthesized according to General Procedure D using 10-fluoro-4-propyl-1-thioxo-2,4-dihydrobenzofuro[2,3-e][1,2,4]triazolo[4,3-a]pyrimidin-5 (1H)-one (9, compound 41 in {Park, 2023 #3387}) as starting material (50%). ¹H NMR (400 MHz, DMSO-d₆): 8.11 (s, 1H), 7.91 (d, J=8.4 Hz, 1H), 7.79-7.84 (m, 1H), 7.49-7.54 (m, 1H), 4.19 (t, J=7.0 Hz, 2H), 3.84 (s, 3H), 1.75 (m, 1H), 0.91 (t, J=7.4 Hz, 3H); 19F NMR (376 MHz, DMSO-d₆): 6-101.39; HRMS m/z (M+H) for C₁₈H₁₅FN₇O₄S calculated 444.0890, found 444.0897.

1-((1-Methyl-4-nitro-1H-imidazol-5-yl)thio)-4-propylthieno[2,3-e][1,2,4]triazolo[4,3-a]pyrimidin-5 (4H)-one, 12. 1-((1-Methyl-4-nitro-1H-imidazol-5-yl)thio)-4-propylthieno[2,3-e][1,2,4]triazolo[4,3-a]pyrimidin-5 (4H)-one was synthesized according to General Procedure D using 4-propyl-1-thioxo-2,4-dihydrothieno[2,3-e][1,2,4]triazolo[4,3-a]pyrimidin-5 (1H)-one (11, compound 42 in {Park, 2023 #3387}) as starting material (40%). ¹H NMR (400 MHz, DMSO-d₆): δ 8.41 (d, J=5.5 Hz, 1H), 8.19 (d, J=5.2 Hz, 1H), 8.14 (s, 1H), 4.14 (t, J=7.4 Hz, 2H), 3.86 (s, 3H), 1.75 (m, 1H), 0.91 (t, J=7.4 Hz, 3H); HRMS m/z (M+H) for C₁₄H₁₄N₇O₃S₂ calculated 392.0600, found 392.0601.

7-Chloro-1-((1-methyl-4-nitro-1H-imidazol-5-yl)thio)-4-propylthieno[2,3-e][1,2,4]triazolo[4,3-a]pyrimidin-5 (4H)-one, 14. 7-Chloro-1-((1-methyl-4-nitro-1H-imidazol-5-yl)thio)-4-propylthieno[2,3-e][1,2,4]triazolo[4,3-a]pyrimidin-5 (4H)-one was synthesized according to General Procedure D using 7-chloro-4-propyl-1-thioxo-1,2-dihydrothieno[2,3-e][1,2,4]triazolo[4,3-a]pyrimidin-5 (4H)-one (13, compound 45 in {Park, 2023 #3387}) as starting material (55%). ¹H NMR (400 MHz, DMSO-d₆): δ 8.29 (s, 1H), 8.14 (s, 1H), 4.12 (t, J=7.2 Hz, 2H), 3.89 (s, 3H), 1.73 (m, 2H), 0.90 (t, J=7.4 Hz, 3H); HRMS m/z (M+H) for C₁₄H₁₃ClN₇O₃S₂ calculated 426.0210, found 426.0212.

7-(tert-Butyl)-1-((1-methyl-4-nitro-1H-imidazol-5-yl)thio)-4-propylthieno[2,3-e][1,2,4]triazolo[4,3-a]pyrimidin-5 (4H)-one, 16. 7-(tert-Butyl)-1-((1-methyl-4-nitro-1H-imidazol-5-yl)thio)-4-propylthieno[2,3-e][1,2,4]triazolo[4,3-a]pyrimidin-5 (4H)-one was synthesized according to General Procedure D using 7-(tert-butyl)-4-propyl-1-thioxo-2,4-dihydrothieno[2,3-e][1,2,4]triazolo[4,3-a]pyrimidin-5 (1H)-one (15, compound 46 in {Park, 2023 #3387}) as starting material (53%). ¹H NMR (400 MHz, DMSO-d₆): δ 8.10 (s, 1H), 7.99 (s, 1H), 4.13 (t, J=7.2 Hz, 2H), 3.84 (s, 3H), 1.73 (m, 1H), 0.90 (t, J=7.4 Hz, 3H); HRMS m/z (M+H) for C₁₈H₂₂N₇O₃S₂ calculated 448.1226, found 448.1221.

1-((1-Methyl-4-nitro-1H-imidazol-5-yl)thio)-4-propylbenzo[4,5]thieno[2,3-e][1,2,4]triazolo[4,3-a]pyrimidin-5 (4H)-one, 18. This compound was synthesized according to General Procedure D using 4-propyl-1-thioxo-2,4-dihydrobenzo[4,5]thieno[2,3-e][1,2,4]triazolo[4,3-a]pyrimidin-5 (1H)-one (17, compound 48 in {Park, 2023 #3387}) as starting material (52%). ¹H NMR (400 MHz, DMSO-d₆): δ 9.42 (d, J=8.2 Hz, 1H), 8.31 (d, J=7.9 Hz, 1H), 8.12 (s, 1H), 7.66-7.75 (m, 2H), 4.20 (t, J=7.6 Hz, 2H), 3.80 (s, 3H), 1.79 (m, 1H), 0.92 (t, J=7.5 Hz, 3H); HRMS m/z (M+H) for C₁₈H₁₆N₇O₃S₂ calculated 442.0756, found 442.0762.

Reagents: (a) CSCl2, TEA, THF, 0° C. to rt, 3 h, crude; (b) (cyclopropyl)methyl amine, THF, reflux, overnight, crude; (c) KOH, EtOH, reflux, 3 hr, 80%; (d) N₂H₄, EtOH, 80° C., overnight, crude; (e) CS2, KOH, EtOH, 80° C., overnight, 6%.

Methyl 5-chloro-3-isothiocyanatothiophene-2-carboxylate, 29. To a solution of methyl 3-amino-5-chlorothiophene-2-carboxylate hydrochloride (25, 1.00 g, 4.38 mmol) in dry THF (50 mL) at 0° C. under a nitrogen atmosphere was added triethylamine (2.5 mL, 17.6 mmol), and thiophosgene (410 μL, 5.27 mmol). The resulting mixture was allowed to warm to room temperature and stirred for 3 h. The reaction was diluted with water and the THF removed in vacuo. The resulting mixture was extracted three times with ethyl acetate and the organic layers were combined, washed with water and brine, and dried over anhydrous sodium sulfate. The organic extract was decanted and concentrated in vacuo. The crude product (29) was used without further purification.

6-Chloro-3-(cyclopropylmethyl)-2-thioxo-2,3-dihydrothieno[3,2-d]pyrimidin-4 (1H)-one, 33. To a solution of 29 (1.00 g, 4.41 mmol) in dry THF (45 mL) was added cyclopropylmethyl amine (420 μL, 4.85 mmol) at room temperature. The reaction was then set-up for reflux and stirred overnight. The mixture was then cooled to room temperature and concentrated in vacuo. The residue was then redissolved in anhydrous ethanol (40 mL) and (445 mg, 7.93 mmol) of potassium hydroxide was added. The mixture was set-up for reflux and stirred for 3 h. After completion of the reaction, the mixture was diluted with water and the pH was adjusted to 1 with 10 N hydrochloric acid. The resulting mixture was extracted three times with ethyl acetate. The organic layers were combined, washed with water and brine, and dried over anhydrous sodium sulfate. The organic extract was decanted and concentrated in vacuo and the residue purified by silica gel column chromatography (hexane:ethyl acetate=1:0 to 3:1) to afford the compound 33 (964 mg, 80%). ¹H NMR (400 MHz, DMSO) δ 14.19 (s, 1H), 8.90 (s, 1H), 3.90 (d, J=7.1 Hz, 2H), 1.34-1.22 (m, 1H), 0.58-0.29 (m, 4H); MS (ESI, m/z) 273.0 [M+H]+; ESI-HRMS calcd. m/z for C₁₀H₁₀ClN₂OS₂ 272.9923, found 272.9924.

7-Chloro-4-(cyclopropylmethyl)-1-thioxo-2,4-dihydrothieno[2,3-e][1,2,4]triazolo[4,3-a]pyrimidin-5 (1H)-one, 19. To a solution of 33 (100 mg, 0.367 mmol) in 6 mL of anhydrous ethanol was added anhydrous hydrazine (81 μL, 2.57 mmol) at room temperature. The reaction was sealed in a pressure tube and heated to 80° C. to stir overnight. Then, the reaction was cooled, and the mixture was dried with strong stirring and a flow of nitrogen gas. The crude residue was then redissolved with 6 mL anhydrous ethanol, and (61 mg, 1.10 mmol) of potassium hydroxide and (222 μL, 3.67 mmol) of carbon disulfide were added. The pressure tube was once again sealed, and the reaction was allowed to stir overnight at 80° C. The reaction was cooled to room temperature and then the pH was adjusted to 1 with 10N hydrochloric acid. The mixture was then extracted three times with ethyl acetate. The organic layers were combined, washed with water and brine, and dried over anhydrous sodium sulfate. The organic extract was decanted and concentrated in vacuo and the residue purified by silica gel column chromatography (hexane:ethyl acetate=1:0 to 20:3) to afford the compound 19 (6 mg, 6%). ¹H NMR (400 MHz, DMSO) § 14.20 (s, 1H), 8.92 (s, 1H), 3.90 (d, J=7.1 Hz, 2H), 1.29 (q, J=7.6 Hz, 1H), 0.54-0.35 (m, 4H); HRMS m/z (M+H) for C₁₁H₉ClN₄OS₂ calculated 312.9985, found 312.9982.

7-Chloro-4-(cyclopropylmethyl)-1-((1-methyl-4-nitro-1H-imidazol-5-yl)thio) thieno[2,3-e][1,2,4]triazolo[4,3-a]pyrimidin-5 (4H)-one, 20. 7-Chloro-4-(cyclopropylmethyl)-1-((1-methyl-4-nitro-1H-imidazol-5-yl)thio) thieno[2,3-e][1,2,4]triazolo[4,3-a]pyrimidin-5 (1H)-one was synthesized according to General Procedure D using 7-chloro-4-(cyclopropylmethyl)-1-thioxo-2,4-dihydrothieno[2,3-e][1,2,4]triazolo[4,3-a]pyrimidin-5 (1H)-one (19) as the starting material (78%). ¹H NMR (400 MHz, DMSO-d₆): δ 8.29 (s, 1H), 8.13 (s, 1H), 4.05 (d, J=7.1 Hz, 2H), 3.90 (s, 3H), 1.46 (s, 9H), 1.30 (m, 1H), 0.47 (m, 4H); HRMS m/z (M+H) for C₁₅H₁₂ClN₇O₃S₂ calculated 438.0210, found 438.0217.

N-(3-(7-Fluoro-5-oxo-1-thioxo-1,2-dihydro-[1,2,4]triazolo[4,3-a]quinazolin-4 (5H)-yl) propyl)-1-(5-(2-oxohexahydro-1H-thieno[3,4-d]imidazol-4-yl) pentanamido)-3,6,9,12-tetraoxapentadecan-15-amide, 22. This compound was synthesized as follows. To a solution of 1-amino-N-(3-(7-fluoro-5-oxo-1-thioxo-1,2-dihydro-[1,2,4]triazolo[4,3-a]quinazolin-4 (5H)-yl) propyl)-3,6,9,12-tetraoxapentadecan-15-amide (54 mg, 0.01 mmol, compound 94 in {Alverez, 2020 #3186}) in DMF (0.5 mL). Biotin N-hydroxysuccinimide ester (4.0 mg, 0.012 mmol) and TEA (0.0017 mL) were added, and the reaction mixture was stirred at RT overnight. The reaction mixture was concentrated, and the product was purified by column chromatography (DCM/MeOH 90:10) as light-yellow solid. 1H NMR (400 MHz, methanol-d₄) δ 10.50 (dd, J=9.3, 4.6 Hz, 1H), 7.97 (dd, J=8.5, 3.1 Hz, 1H), 7.62 (ddd, J=9.3, 7.7, 3.1 Hz, 1H), 4.48 (dd, J=8.0, 4.8 Hz, 1H), 4.38-4.14 (m, 2H), 3.73 (t, J=6.0 Hz, 1H), 3.61 (dd, J=10.5, 3.8 Hz, 8H), 3.52 (t, J=5.5 Hz, 1H), 3.27-3.10 (m, 2H), 2.92 (ddd, J=12.6, 7.2, 5.0 Hz, 1H), 2.78-2.57 (m, 1H), 2.46 (t, J=6.0 Hz, 1H), 2.21 (t, J=7.3 Hz, 2H), 2.02 (p, J=6.9 Hz, 1H), 1.84-1.50 (m, 2H), 1.52-1.22 (m, 3H), 1.03 (t, J=7.4 Hz, 2H). HRMS m/z (M+H) for C₃₃H₄₇FN₈O₈S₂ calculated 766.9054, found 767.3028.

### Example 2

This example illustrates methods of screening and testing the compounds of the invention. For virtual screening, the deposited structure of human Plk1 PBD in complex with Allopole-A (PDB: 8CRC) was used. To verify that the structure allows for accurate prediction of binding, a test database of 6,985,997 on-the shelf compounds from ChemSpace (Kiev, Ukraine) was docked into Allopole pocket using Rapid Docking GPU Engine (RIDGE) from Molsoft (Molsoft L.L.C., San Diego, CA) and NIH Biowulf cluster supercomputer. Docking produced 37,729 potential hits that have been redocked with high thoroughness using VLS mode of ICM-Pro software (Molsoft L.L.C., San Diego, CA) by running 800 parallel processes on Biowulf. Two types of scores have been computed: LE score based on molecular mechanics with generalized Born and surface area (GBSA/MM), and RTCNN, a Radial Convolutional Neural Net score trained to recognize native-like complexes that were deposited in Protein Database. Eight highest scoring compounds (Nad-1-Nad-8) have been supplied by ChemSpace and tested for binding using fluorescence polarization and ELISA assays.

After verification of the accuracy of the computational predictions using PDB: 8CRC structure, it was used for RIDGE screening of diversity sets of compounds from Enamine (48 million), SAVI (5 million) and WuXiAppTec (5 million). Only SAVI diversity set produced better scoring compounds than original ChemSpace screen.

To cover larger chemical space in searches, ligand-based screens of conformers for 15.5 billion compounds have been screened using Rapid Isostere Discovery Engine (Molsoft L.L.C.) and local Linux server equipped with 4 GPUs and 54 CPUs. 1.7 billon compounds were from SAVI (Synthetically accessible virtual inventory, NCI), 5.5 billion from Freedom (ChemSpace), 6.5 billion from REAL (Enamine) and 1.7 billion from Galaxi (WuXiAppTec). Conformers for the databases have been generated on NIH HPC Biowulf using thousands of CPUs (30 million CPU Hours in total). Four lead compounds have been used as templates in RIDE searches: Allopole, Nad-1 and Nad-6 and best SAVI hit. Potential hits from RIDE searches have been redocked with high thoroughness using VLS mode of ICM-Pro software. Compounds CIL-1-CIL-18 have been selected for further testing as they had the lowest RTCNN an LE scores. This Example illustrates the anti-PBD1 activity of 4-derived heterocyclic inhibitors in accordance with an aspect of the invention, as determined by ELISA-based and L363 cell-based assays.

ELISA-based PBD1-binding inhibition assay. The assay, which is designed to determine the ability of a compound to inhibit the interaction between the full-length Plk1 and a biotinylated PBIP1 phospho-T78 peptide (i.e., Biotin-Ahx-CETFDPPLHSpTAI-NH2) {Kang, 2006 #3234; Yun, 2009 #2399}, was performed essentially as described previously {Yun, 2009 #2399}. The source of the full-length Plk1 was the total lysates prepared from HEK293A cells infected with an adenovirus expressing human influenza hemagglutinin (HA) and EGFP-fused Plk1. Data are reported as IC₅₀ to indicate the concentration producing 50% inhibition of the phosphopeptide binding. The reaction products were measured at 450 nm by using a Perkin-Elmer Enspire Multimode Plate reader (PerkinElmer, Inc., Boston, MA). All the data were obtained from more than three independent experiments and were analyzed by GraphPad (San Diego, CA) Prism software version 7.

In the following, PBD1-3 fluorescence polarization (FP) binding assays were carried out for determining the Plk1 specificity. FP assays were carried out essentially as described previously {Liu, 2011 #2790; Qian, 2014 #1416}. All samples were analyzed approximately 30 min after reaction in a 384-well format using the SpectraMax Paradigm multi-mode microplate detection platform (Molecular Devices). Data were analyzed after carrying out at least three independent experiments using GraphPad Prism software version 7.

TABLE 1
Anti-PBD1 activity of 4-derived heterocyclic inhibitors determined by
ELISA-based and L363 cell-based assays.

| | | ELISA (IC₅₀) | Cell efficacy |
|---|---|---|---|
| | | Mean ± SEM | (GI₅₀) |
| Compound | Structure | (μM)^{a} | Mean ± SD (μM)^{b} |
| 1, PLHST | 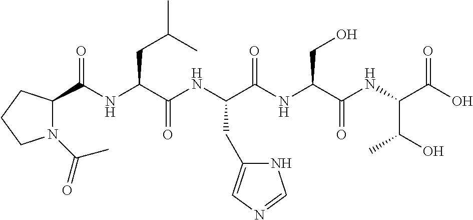 [SMILES: CC(=O)N1CCC[C@H]1C(=O)N[C@@H](CC(C)C)C(=O)N[C@@H](Cc1cnc[nH]1)C(=O)N[C@@H](CO)C(=O)N[C@H](C(=O)O)[C@@H](C)O] | >50 | N.S. |
| 2, PLHSpT | 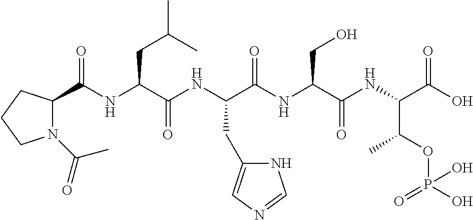 [SMILES: CC(=O)N1CCC[C@H]1C(=O)N[C@@H](CC(C)C)C(=O)N[C@@H](Cc1cnc[nH]1)C(=O)N[C@@H](CO)C(=O)N[C@H](C(=O)O)[C@@H](C)OP(=O)(O)O] | 13.97 ± 1.00 | N.S. |
| 3, NCK149* | 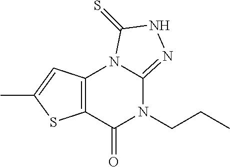 [SMILES: CCCn1c(=O)c2sc(C)cc2n2c(=S)[nH]nc12] | 2.15 ± 0.13 (6) | N.S. |
| 4, NCK173 (80)* (prodrug of 3) | 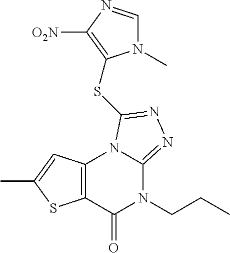 [SMILES: CCCn1c(=O)c2sc(C)cc2n2c(Sc3c([N+](=O)[O-])ncn3C)nnc12] | >50 | 4.88 ± 0.10 (low solubility) |
| 5, NCK181 | 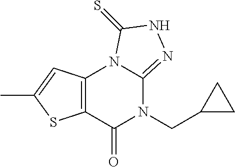 [SMILES: Cc1cc2c(s1)c(=O)n(CC1CC1)c1n[nH]c(=S)n21] | 1.66 ± 0.04 (4) | N.S. |
| 6, NCK182 (prodrug of 5) | 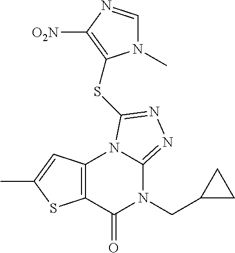 [SMILES: Cc1cc2c(s1)c(=O)n(CC1CC1)c1nnc(Sc3c([N+](=O)[O-])ncn3C)n21] | >50 | 3.13 ± 0.15 (low solubility) |
| 8, NCK183 (prodrug of 7 = 40 in Park) | 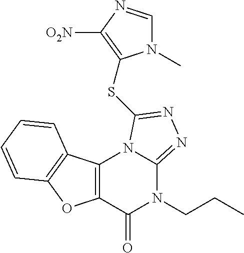 [SMILES: CCCn1c(=O)c2oc3ccccc3c2n2c(Sc3c([N+](=O)[O-])ncn3C)nnc12] | >50 | 8.31 ± 0.56 |
| 10, NCK184 (prodrug of 9 = 41 in Park) | 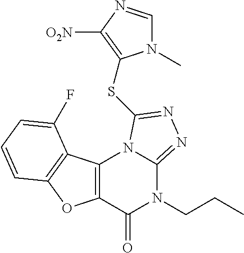 [SMILES: CCCn1c(=O)c2oc3cccc(F)c3c2n2c(Sc3c([N+](=O)[O-])ncn3C)nnc12] | >50 | 7.53 ± 0.43 |
| 12, NCK185 (prodrug of 11 = 42 in Park) | 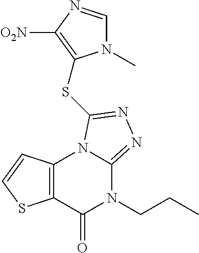 [SMILES: CCCn1c(=O)c2sccc2n2c(Sc3c([N+](=O)[O-])ncn3C)nnc12] | >50 | 8.40 ± 0.74 (moderate solubility) |
| 14, NCK186 (prodrug of 13 = 45 in Park) | 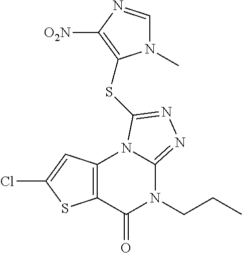 [SMILES: CCCn1c(=O)c2sc(Cl)cc2n2c(Sc3c([N+](=O)[O-])ncn3C)nnc12] | >50 | 6.04 ± 0.17 |
| 16, NCK187 (prodrug of 15 = 46 in Park) | 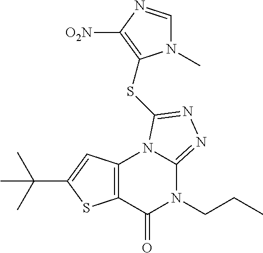 [SMILES: CCCn1c(=O)c2sc(C(C)(C)C)cc2n2c(Sc3c([N+](=O)[O-])ncn3C)nnc12] | >50 | 4.19 ± 0.15 (moderate solubility) |
| 18, NCK188 (prodrug of 17 = 48 in Park) | 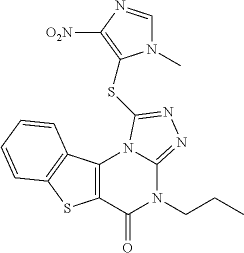 [SMILES: CCCn1c(=O)c2sc3ccccc3c2n2c(Sc3c([N+](=O)[O-])ncn3C)nnc12] | >50 | 7.05 ± 0.27 |
| 19, NCK189 | 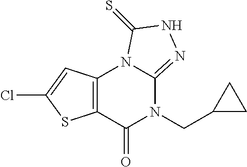 [SMILES: O=c1c2sc(Cl)cc2n2c(=S)[nH]nc2n1CC1CC1] | 1.40 ± 0.03 | N.S. |
| 20, NCK190 (prodrug of 19) | 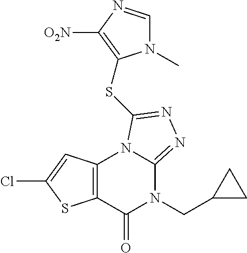 [SMILES: Cn1cnc([N+](=O)[O-])c1Sc1nnc2n(CC3CC3)c(=O)c3sc(Cl)cc3n12] | >25 | 4.09 ± 0.14 |
| 21, NCK49** | 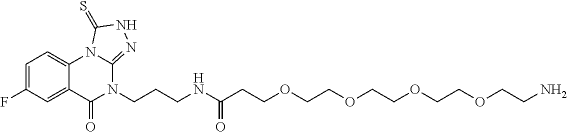 [SMILES: NCCOCCOCCOCCOCCC(=O)NCCCn1c(=O)c2cc(F)ccc2n2c(=S)[nH]nc12] | 5.36 ± 0.40 | N.D. |
| 22, NCK195 | 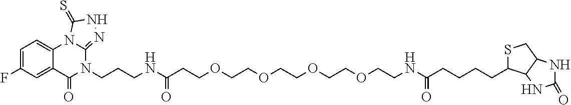 [SMILES: O=C(CCCCC1SCC2NC(=O)NC21)NCCOCCOCCOCCOCCC(=O)NCCCn1c(=O)c2cc(F)ccc2n2c(=S)[nH]nc12] | 9.32 ± 0.17 | N.D. |

*Data from Park, J. E., et al {Park, 2023 #3387}. **Data from Alverez C. N., et al {Alverez, 2020 #3186}. N. S., Not significant; N. D., Not determined. ^{a} IC₅₀ values determined from at least three independent experiments except as noted in parentheses. b A colorimetric MTS cell viability assay carried out using multiple myeloma-derived L363 cells. The concentration inhibiting 50% cell growth (GI₅₀) was determined from at least three independent experiments.

The structural formulas of drug molecules 7, 9, 11, 13, 15, 17, and 19 are set forth in Table 2.

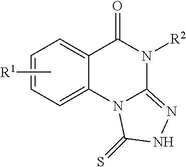

TABLE 2
Drug compounds in accordance with an aspect of the invention

| Drug | | |
|---|---|---|
| (compound number) | R¹ | R² |
| 7 | H | 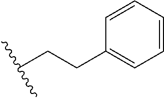 [SMILES: *CCc1ccccc1] |
| 9 | 9-F | 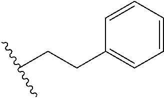 [SMILES: *CCc1ccccc1] |
| 11 | 9-F | 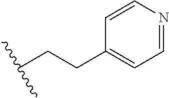 [SMILES: *CCc1ccncc1] |
| 13 | 9-F | ethyl |
| 15 | 9-F | n-propyl |
| 17 | 7-OMe | n-propyl |
| 19 | 7-F | 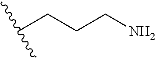 [SMILES: *CCCN] |

Table 3 sets forth the activities of some of the compounds in an aspect of the invention.

TABLE 3
FP-based inhibition assays against Plk1-3 PBDs.

| | | PBD1 | Plk2 PBD | Plk3 PBD |
|---|---|---|---|---|
| | Respective | IC₅₀ ± SD | IC₅₀ ± SD | IC₅₀ ± SD |
| Compound | prodrug | [nM] | [nM] | [nM] |
| 2, PLHSpT | | 35.02 ± 5.8 | N.S. | N.S. |
| 3, NCK149 | 4. NCK173 | 10.17 ± 1.5 | N.S. | N.S. |
| 5, NCK181 | 6, NCK182 | 12.83 ± 2.6 | N.S. | N.S. |
| 19, Allopole-A | 20, NCK190 | 2.53 ± 0.18 | N.S. | N.S. |

N. S., Not significant. IC₅₀ values determined from at
least three independent experiments.

### Example 3

This Example illustrates the conversion of Allopole to Allopole-A under various buffer conditions, RT, 1 h (JE), as set forth in Table 4

TABLE 4
Conversion of prodrugs

| | Compounds detected | | | | |
|---|---|---|---|---|---|
| Buffers | | Allopole A | Allopale-A (-S) | Allopole (-Cl) | Allopole |
| 3 | pk area | 9.61E+08 | 6.64E+06 | 4.85E+04 | 7.45E+08 |
| | umol/L | 1.3 | 0.9 | 0.0 | 97.9 |
| | % of | 1.3 | 0.9 | 0.8 | 97.9 |
| | total | | | | |
| | pk area | 1.38E+09 | 5.81E+07 | 8.898+05 | 8.76E+08 |
| 1 + 5 mM | umol/L | 95.4 | 4.0 | 0.5 | 0.1 |
| GSH | % of | 95.4 | 4.0 | 0.5 | 0.1 |
| | total | | | | |
| 2 | pk area | 4.90E+06 | 1.05E+07 | 2.425:04 | 6.28E+08 |
| | umol/L | 0.8 | 1.6 | 0.0 | 97.6 |
| | % of | 0.8 | 1.6 | 0.0 | 97.6 |
| | total | | | | |
| 2 + 5 mM | pk area | 5.81E+08 | 1.63E+08 | 2.578+06 | 2.41E+03 |
| GSH | umol/L | 77.9 | 21.8 | 0.3 | 0.0 |
| | % of | 77.9 | 21.8 | 0.3 | 0.0 |
| | total | | | | |
| 3 | pk area | 1.56E+06 | 4.21E+06 | 0.00E+00 | 7.80E+17 |
| | umol/L | 1.9 | 5.0 | 0.0 | 93.1 |
| | % of | 1.9 | 5.0 | 0.0 | 93.1 |
| | total | | | | |
| 3 + 5 mM | pk area | 1.89E+08 | 9.21E+07 | 1.11E+06 | 9.35+04 |
| GSN | umol/L | 66.9 | 32.7 | 0.4 | 0.0 |
| | % of | 66.9 | 32.7 | 0.4 | 0.0 |
| | total | | | | |

100 uM Allopole was incubated in the indicated buffer
in the absence or presence of 5 mM GSH at RT for 1 h.
Buffer #1: 20 mM Tris 8.0, 50 mM NaCl, 1 mM EDTA, 1% DMSO
Buffer #2: 1x PBS pH = 7.4, 1 mM EDTA, 1% DMSO
Buffer #3: 20 mM HEPES pH = 7.0, 50 mM NaO, 1 mM EDTA, 1 % DMSO

Mass spectroscopy analyses showed rapid conversion of the prodrug Allopole to the active Allopole-A form. Samples were prepared by incubating Allopole in the indicated buffer conditions without or with 5 mM GSH for 1 h, at RT. Note that in the absence of GSH, Allopole remains stable.

Off-target Ki values for selected compounds described herein are set forth in Table 5.

TABLE 5
Off-target Ki values

| | Allopole-A | | Allopole | |
|---|---|---|---|---|
| | Primary screenª | Secondary screen^{b} Ki | Primary screenª | Secondary screen^{b} Ki |
| Receptor | AVE (% inhib) ± SD | (μM) | AVE (% inhib) ± SD | (μM) |
| 5-HT1A | -20.0 ± 27.4 | >10 | -12.8 ± 19.7 | >10 |
| 5-HT1B^{d} | 17.7 ± 42.2 | >10 | 60.8 ± 40.9 | >10 |
| 5-HT1D | 22.5 ± 25.6 | >10 | 32.8 ± 27.1 | >10 |
| 5-HT1E | 31.3 ± 7.6 | >10 | 22.2 ± 10.5 | >10 |
| 5-HT2A^{d} | 37.0 ± 10.4 | >10 | 52.4 ± 11.8 | 9.2 |
| 5-HT2B | -17.7 ± 11.2 | >10 | -0.5 ± 5.0 | >10 |
| 5-HT2C^{d} | -18.3 ± 22.0 | >10 | 59.5 ± 3.0 | 1.3 |
| 5-HT3 | -3.8 ± 3.1 | >10 | -0.8 ± 4.6 | >10 |
| 5-HT5A | -1.4 ± 14.4 | >10 | 24.5 ± 9.3 | >10 |
| 5-HT6 | -5.0 ± 11.7 | >10 | 21.9 ± 15.6 | >10 |
| 5-HT7A | -88.1 ± 20.8 | >10 | 14.9 ± 9.9 | >10 |
| Alpha1A | -9.4 ± 18.0 | >10 | 40.6 ± 10.5 | >10 |
| Alpha1B | -7.7 ± 9.7 | >10 | -1.6 ± 9.5 | >10 |
| Aipha1D | -9.9 ± 23.4 | >10 | -15.1 ± 19.8 | >10 |
| Alpha2A | -6.3 ± 7.9 | >10 | 9.4 ± 4.6 | >10 |
| Alpha2B | -6.4 ± 7.1 | >10 | 8.3 ± 16.4 | >10 |
| Alpha2C | 10.6 ± 4.2 | >10 | 6.4 ± 7.5 | >10 |
| Beta1^{d} | -6.2 ± 19.4 | >10 | 67.3 ± 49.1 | >10 |
| Beta2 | -2.1 ± 19.7 | >10 | 0.1 ± 10.6 | >10 |
| Beta3 | 17.9 ± 8.6 | >10 | 30.8 ± 22.8 | >10 |
| BZP Rat brain site | 2.3 ± 21.8 | >10 | 9.5 ± 7.6 | >10 |
| D1 | -17.2 ± 25.0 | >10 | 21.5 ± 6.8 | >10 |
| D2 | -27.4 ± 13.1 | >10 | 3.6 ± 6.6 | >10 |
| D3 | -7.0 ± 21.7 | >10 | 12.8 ± 12.8 | >10 |
| D4 | -24.3 ± 4.4 | >10 | 12.6 ± 4.9 | >10 |
| D5 | 28.7 ± 10.7 | >10 | 20.9 ± 9.5 | >10 |
| DAT^{d} | 43.9 ± 13.0 | >10 | 54.0 ± 10.7 | >10 |
| DOR | -13.5 ± 6.8 | >10 | -1.0 ± 4.8 | >10 |
| GABAA | 35.7 ± 11.1 | >10 | 33.3 ± 4.2 | >10 |
| H1 | 11.5 ± 2.8 | >10 | 35.8 ± 11.9 | >10 |
| H2 | -27.4 ± 17.1 | >10 | -16.4 ± 21.5 | >10 |
| H3 | -18.5 ± 3.8 | >10 | -12.5 ± 7.9 | >10 |
| H4 | -4.5 ± 11.4 | >10 | 8.2 ± 3.1 | >10 |
| KOR | -0.3 ± 8.7 | >10 | 3.7 ± 4.1 | >10 |
| M1^{d} | 39.3 ± 10.7 | >10 | 50.7 ± 26.1 | >10 |
| M2 | 21.1 ± 19.7 | >10 | 9.3 ± 19.3 | >10 |
| M3 | 7.5 ± 45.6 | >10 | 48.3 ± 21.9 | >10 |
| M4 | -8.8 ± 4.8 | >10 | -8.7 ± 9.5 | >10 |
| M5 ^{d} | 130.2 ± 3.0 | >10 | 95.0 ± 7.4 | >10 |
| MOR | 30.8 ± 47.5 | >10 | 40.6 ± 51.0 | >10 |
| NET | -39.3 ± 20.6 | >10 | -56.8 ± 10.5 | >10 |
| PBR | 5.3 ± 8.3 | >10 | -2.1 ± 5.3 | >10 |
| SERT | -28.9 ± 28.7 | >10 | -56.0 ± 13.9 | >10 |
| Sigma 1 | 2.0 ± 21.4 | >10 | 41.8 ± 11.9 | >10 |
| Sigma 2 | 40.6 ± 9.5 | >10 | 40.7 ± 5.8 | >10 |

^{a}Primary screening has been tested with 10 mM sample -
^{b}Secondary screening result is Ki or >10 mM. - (Ki or >10 uM)
^{c}Data of the secondary screen of Allopole-A has been measured. -
^{d}Data of the secondary screen of Allopole has been measured. -

Antibodies used herein are set forth in Table 6.

TABLE 6
Antibodies

| Antibodies | Species | Source | Cat # | Exp. dilution |
|---|---|---|---|---|
| Primary antibodies | | | | |
| Anti-PB 1P | Rabbit | {Kang, 2006 #3234 | Lab supply | 1:500 (1B) |
| Anti-Bub1 | Rabbit | {Tang, 2001 #1840} | Lab supply | 1:500 (1B) |
| Anti-BubR1 | Mouse | BD Biosciences | BD612503 | 1:1000 (1B) |
| Anti-Cdc25C (C-20) | Rabbit | Santa Cruz Biotech | sc-327 | 1:1000 (1B) |
| Anti-Cdc25C pT48 | Rabbit | Cell Signaling | #9527 | 1:1000 (1B) |
| [symbol] | Mouse | BD Biosciences | BD610154 | 1:1000 (1B) |
| Anti-TCTP | Rabbit | Cell Signaling | #8441 | 1:1000 (1B) |
| Anti-TCTP pS46 | Rabbit | Cell Signaling | #5251 | 1:1000 (1B) |
| Anti-FLAG (M2) | Mouse | Millipore Sigma | F1804 | 1:1000 (1B) |
| Anti-FLAG M2 Affinity Gei | Mouse | Millipore Sigma | A2220 | |
| Anti-FLAG (M2)-HRP conjugate | Mouse | Millipore Sigma | A8592 | 1:10000 (1B) |
| Anti-Pik1 (F-8) | Mouse | Santa Cruz Biotech | sc-17783 | 1:400 (1B), |
| | | | | 1:100 (1F) |
| Anti-Cep192 (1-647) | Rabbit | {Kim, 2013 #1252} | Lab supply | 1:200 (1F) |
| Anti-Crest | Human | Antibodies Inc | 15-234 | 1:500 (1F) |
| Secondary antibodies (Immunofluorescence) | | | | |
| Anti-mouse 1gG Aiexa | Donkey | Invitrogen | A-21203 | 1:300 (1F) |
| Fluor 594-conjugated | | | | |
| Anti-rabbit 1gG | Donkey | Invitrogen | A-31573 | 1:300 (1F) |
| Alexa Fluor 647- | | | | |
| conjugated | | | | |
| Anti-human 1gG Alexa | Goat | Invitrogen | A-11013 | 1:300 (1F) |
| Fiuor 488-conjugated | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I), a prodrug of formula (II), or a pharmaceutically acceptable salt thereof, 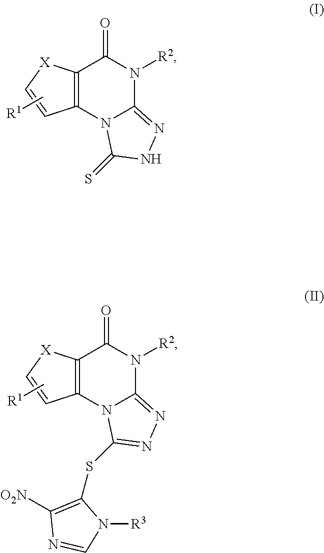
wherein X is O or S;
R¹ is H, halo, C₁-C₆ alkyl, C₃-C₈ cycloalkyl, C₃-C₈ cycloalkyl C₁-C₃ alkyl, 4,5-fused phenyl, or 4,5-fused halophenyl;
R² is halo or C₁-C₆ alkyl; and
R³ is C₁-C₃ alkyl;
or a compound selected from the group consisting of compounds Nad-1 to Nad-8 and CIL-1 to CIL-18: 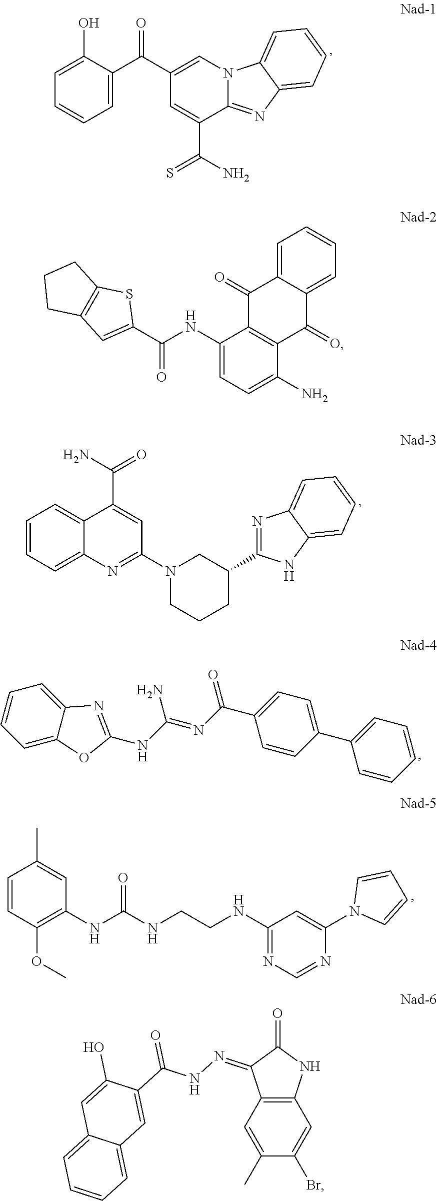 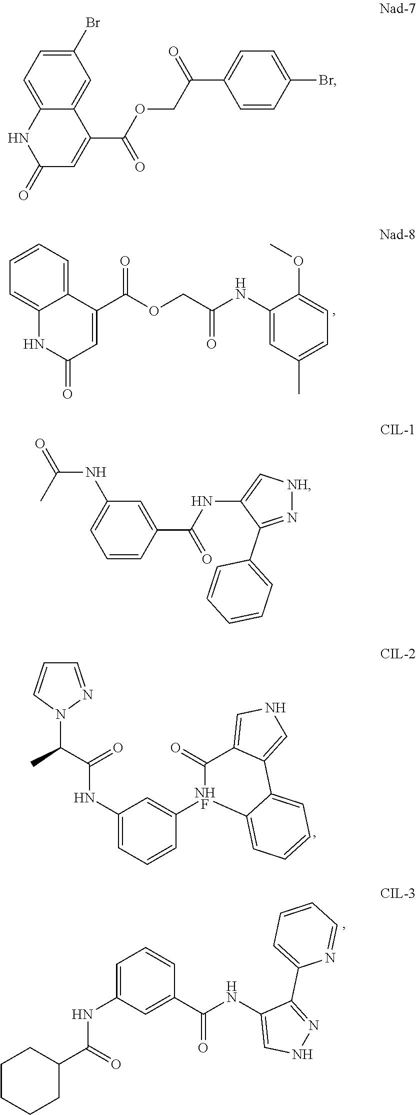 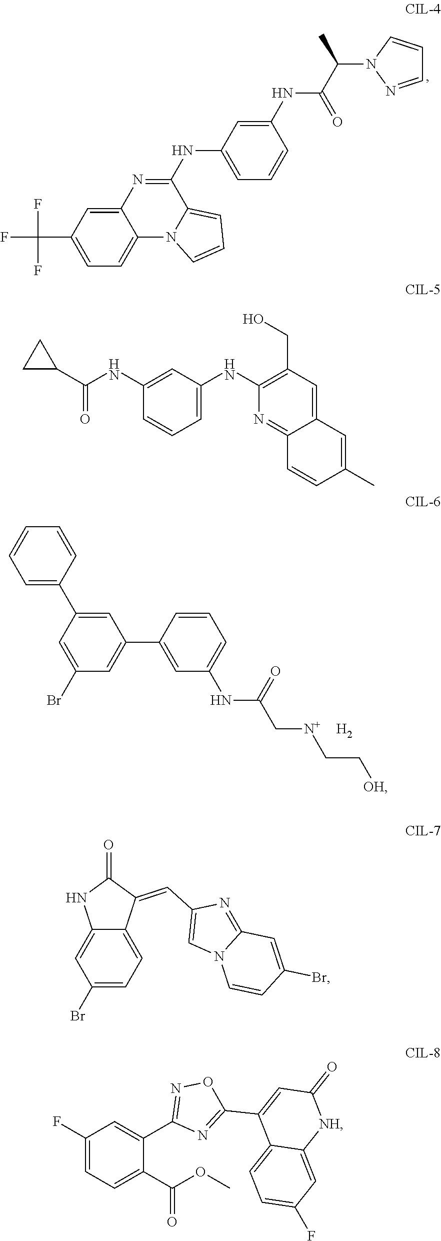 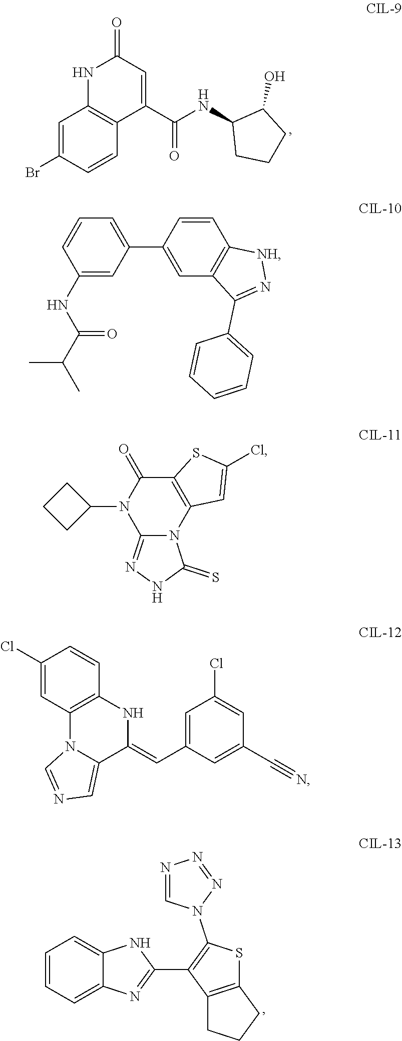 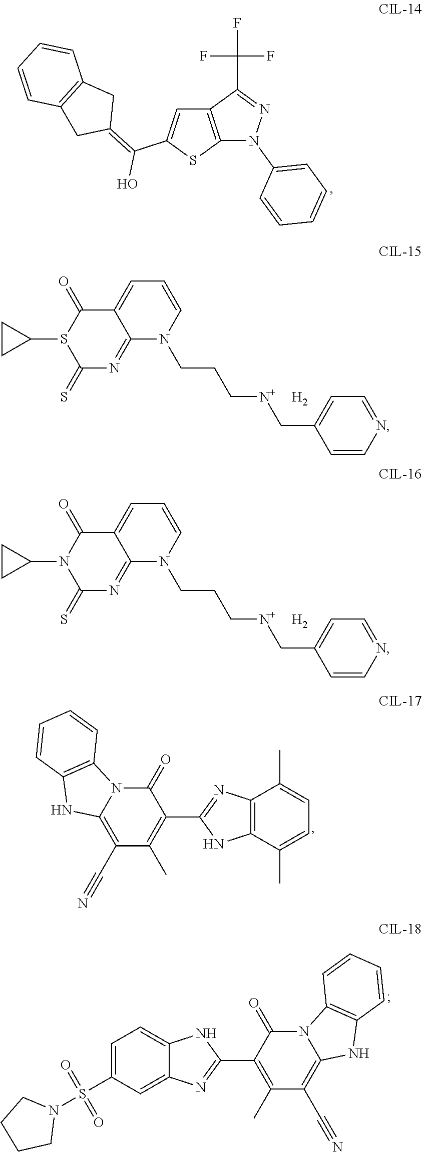
a compound of formula 22, a compound of formula (A), (B), (C), (D), or (E), or a pharmaceutically acceptable salt thereof:

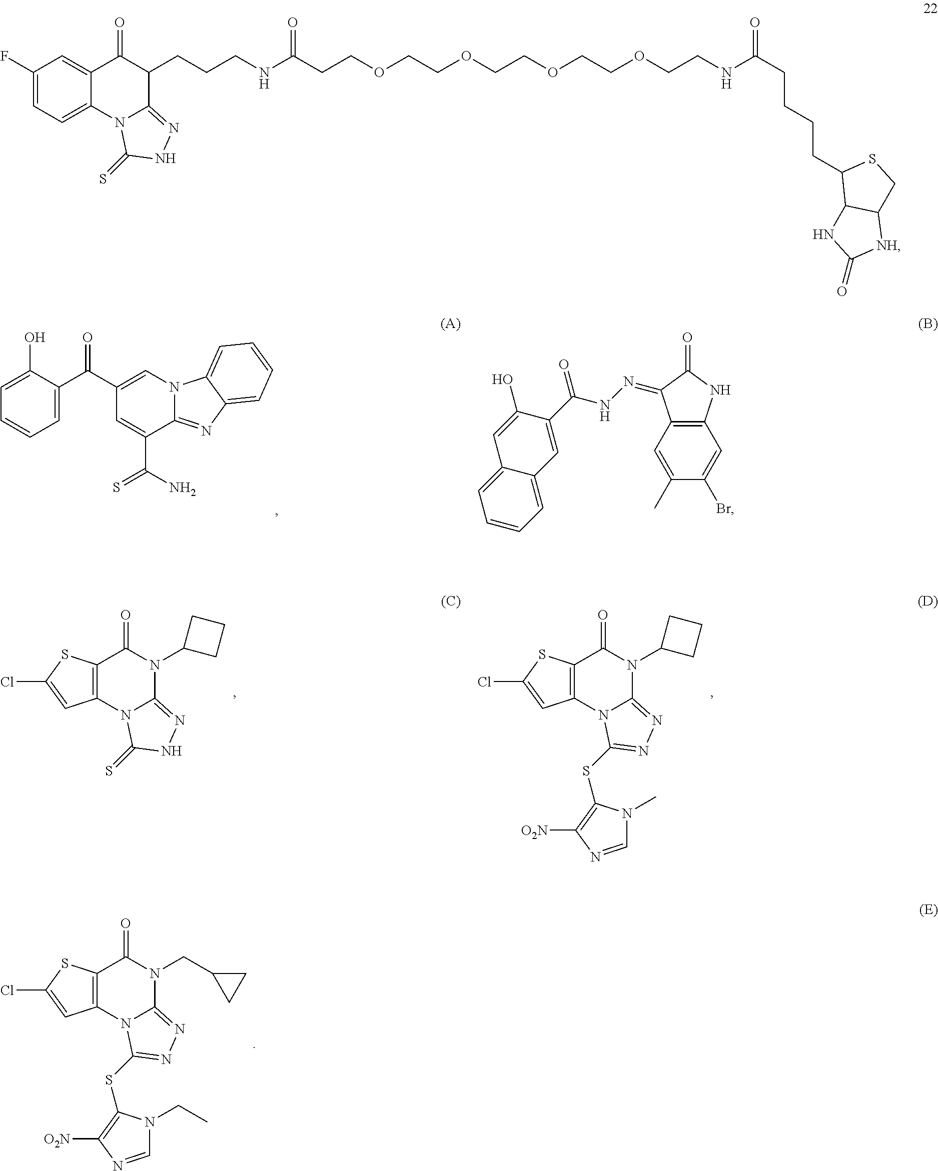 [SMILES: O=C(CCCCC1SCC2NC(=O)NC21)NCCOCCOCCOCCOCCC(=O)NCCCC1C(=O)c2cc(F)ccc2-n2c1n[nH]c2=S ; NC(=S)c1cc(C(=O)c2ccccc2O)cn2c1nc1ccccc12 ; Cc1cc2c(cc1Br)NC(=O)/C2=N/NC(=O)c1cc2ccccc2cc1O ; O=c1c2sc(Cl)cc2n2c(=S)[nH]nc2n1C1CCC1 ; Cn1cnc([N+](=O)[O-])c1Sc1nnc2n(C3CCC3)c(=O)c3sc(Cl)cc3n12 ; CCn1cnc([N+](=O)[O-])c1Sc1nnc2n(CC3CC3)c(=O)c3sc(Cl)cc3n12]

2. The compound or pharmaceutically acceptable salt of claim 1, wherein, in the compound of formula (I) or prodrug of formula (II), X is S.

3. The compound or pharmaceutically acceptable salt of claim 2, wherein R¹ is H, 5-CH₃, 5-Cl, 5-C(CH₃)₃, or 4,5-fused phenyl.

4. The compound or pharmaceutically acceptable salt of claim 2, wherein R² is (CH₂)₂CH₃, CH₂-cyclopropyl, or n-propyl.

5. The compound or pharmaceutically acceptable salt of claim 1, wherein, in the compound of formula (I) or prodrug of formula (II), X is O.

6. The compound or pharmaceutically acceptable salt of claim 5, wherein R¹ is 4,5-fused phenyl or 4,5-fused 4-flurophenyl and R² is n-propyl.

7. The compound or pharmaceutically acceptable salt of claim 1, wherein the compound is one of Nad-1 to Nad-8.

8. The compound or pharmaceutically acceptable salt of claim 1, wherein the compound is one of CIL-1 to CIL-18.

9. The compound or pharmaceutically acceptable salt of claim 1, wherein the compound is the compound of formula 22.

10. The compound or pharmaceutically acceptable salt of claim 1, wherein the compound is a compound of formula (C), (D), or (E):

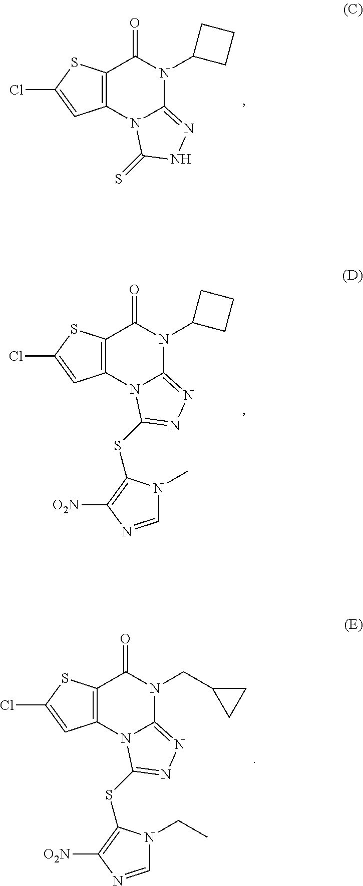 [SMILES: O=c1c2sc(Cl)cc2n2c(=S)[nH]nc2n1C1CCC1 ; Cn1cnc([N+](=O)[O-])c1Sc1nnc2n(C3CCC3)c(=O)c3sc(Cl)cc3n12 ; CCn1cnc([N+](=O)[O-])c1Sc1nnc2n(CC3CC3)c(=O)c3sc(Cl)cc3n12]

11.-15. (canceled)

16. The compound or pharmaceutically acceptable salt of claim 10, wherein the compound is a compound of formula (D).

17. A pharmaceutical composition comprising a compound or salt according to claim 1, and a pharmaceutically acceptable carrier.

18. A pharmaceutical composition comprising a compound or salt according to claim 16, and a pharmaceutically acceptable carrier.

19. A method of treating cancer in a mammal comprising administering to the mammal a compound or salt according to claim 1.

20. A method of treating cancer in a mammal comprising administering to the mammal a compound or salt according to claim 10.

21. A method of treating cancer in a mammal comprising administering to the mammal a compound or salt according to claim 16.

22. The method according to claim 19, wherein the cancer comprises cancer cells that overexpress polo-like kinase 1 (Plk1) relative to normal cells of the same tissue type.

23. The method according to claim 22, wherein the cancer comprises cancer cells of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals.

24. The method according to claim 20, wherein the cancer comprises cancer cells that overexpress polo-like kinase 1 (Plk1) relative to normal cells of the same tissue type.

25. The method according to claim 24, wherein the cancer comprises cancer cells of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals.

26. The method according to claim 21, wherein the cancer comprises cancer cells that overexpress polo-like kinase 1 (Plk1) relative to normal cells of the same tissue type.

27. The method according to claim 26, wherein the cancer comprises cancer cells of the head and neck, eye, skin, mouth, throat, esophagus, chest, bone, lung, colon, sigmoid, rectum, stomach, prostate, breast, ovaries, kidney, liver, pancreas, brain, intestine, heart, or adrenals.
